(19) United States
(12) Patent Application Publication
(10) Pub. No.: US 2008/0194836 A1
(43) Pub. Date: Aug. 14, 2008

(54) Process for Preparing Substituted Phenylpyrazole Ureas

(76) Inventors: Tawfik Gharbaoui, Escondido (CA); Claudia Averbuj, San Diego, CA (US); Marlony Carlos, Santee, CA (US); Edward A. Lally, La Jolla, CA (US); Dipanjan Sengupta, San Diego, CA (US); John Robert Fritch, Romona, CA (US)

Correspondence Address: FISH & RICHARDSON P.C., P.O. BOX 1022, MINNEAPOLIS, MN, 55440-1022, US

(21) Appl. No.: 11/883,043

(22) PCT Filed: Jan. 25, 2006
(86) PCT No.: PCT/US2006/002721

§ 371 Date: Feb. 22, 2008

Related U.S. Application Data
(60) Provisional application No. 60/647,613, filed on Jan. 26, 2005.

(51) Int. Cl. C07D 231/10

(52) U.S. Cl. 548/375.1

(57) ABSTRACT

The present invention is directed to processes for the preparation of substituted phenylpyrazole ureas of Formula (I) that are useful as 5-HT_{2A} serotonin receptor modulators for the treatment of disease.

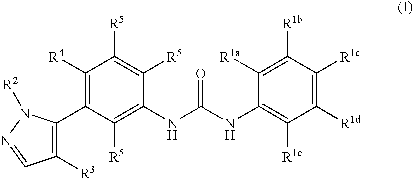

### FIELD OF THE INVENTION

The present invention is directed to processes for the preparation of substituted phenylpyrazole ureas that are useful as 5-HT_{2A} serotonin receptor modulators for the treatment of disease.

### BACKGROUND OF THE INVENTION

G protein-coupled receptors share a common structural motif. All these receptors have seven sequences of between 22 to 24 hydrophobic amino acids that form seven alpha helices, each of which spans the membrane. The transmembrane helices are joined by strands of amino acids having a larger loop between the fourth and fifth transmembrane helix on the extracellular side of the membrane. Another larger loop, composed primarily of hydrophilic amino acids, joins transmembrane helices five and six on the intracellular side of the membrane. The carboxy terminus of the receptor lies intracellularly with the amino terminus in the extracellular space. It is thought that the loop joining helices five and six, as well as, the carboxy terminus, interact with the G protein. Currently, Gq, Gs, Gi and Go are G proteins that have been identified.

Under physiological conditions, G protein-coupled receptors exist in the cell membrane in equilibrium between two different states or conformations: an “inactive” state and an “active” state. A receptor in an inactive state is unable to link to the intracellular transduction pathway to produce a biological response. Changing the receptor conformation to the active state allows linkage to the transduction pathway and produces a biological response.

A receptor may be stabilized in an active state by an endogenous ligand or an exogenous agonist ligand. Recent discoveries such as, including but not exclusively limited to, modifications to the amino acid sequence of the receptor provide means other than ligands to stabilize the active state conformation. These means effectively stabilize the receptor in an active state by simulating the effect of a ligand binding to the receptor. Stabilization by such ligand-independent means is termed “constitutive receptor activation.”

Receptors for serotonin (5-hydroxytryptamine, 5-HT) are an important class of G protein-coupled receptors. Serotonin is thought to play a role in processes related to learning and memory, sleep, thermoregulation, mood, motor activity, pain, sexual and aggressive behaviors, appetite, neurodegenerative regulation, and biological rhythms. Not surprisingly, serotonin is linked to pathophysiological conditions such as anxiety, depression, obsessive-compulsive disorders, schizophrenia, suicide, autism, migraine, emesis, alcoholism, and neurodegenerative disorders. With respect to an anti-psychotic treatment, approaches focused on the serotonin receptors, these types of therapeutics can generally be divided into two classes, the “typical” and the “atypical.” Both have anti-psychotic effects, but the typicals also include concomitant motor-related side effects (extra pyramidal syndromes, e.g. lip-smacking, tongue darting, locomotor movement, etc). Such side effects are thought to be associated with the compounds interacting with other receptors, such as the human dopamine D2 receptor in the nigro-striatal pathway. Therefore, an atypical treatment is preferred. Haloperidol is considered a typical anti-psychotic, and clozapine is considered an atypical anti-psychotic.

Serotonin receptors are divided into seven subfamilies, referred to as 5-HT₁ through 5-HT₇, inclusive. These subfamilies are further divided into subtypes. For example, the 5-HT₂ subfamily is divided into three receptor subtypes: 5-HT_{2A}, 5-HT_{2B}, and 5-HT_{2C}. The human 5-HT_{2C} receptor was first isolated and cloned in 1987, and the human 5-HT_{2A} receptor was first isolated and cloned in 1990. These two receptors are thought to be the site of action of hallucinogenic drugs. Additionally, antagonists to the 5-HT_{2A} and 5-HT_{2C} receptors are believed to be useful in treating depression, anxiety, psychosis, and eating disorders.

Isolation, characterization, and expression of a functional cDNA clone encoding the entire human 5-HT_{1C} receptor (now known as the 5-HT_{2C} receptor) and the entire human 5-HT_{2A} receptor are described in U.S. Pat. Nos. 4,985,352 and 5,661,012, respectively. Mutations of the endogenous forms of the rat 5-HT_{2A} and rat 5-HT_{2C} receptors have been reported to lead to constitutive activation of these receptors (5-HT_{2A}: Casey, C. et al. (1996) Society for Neuroscience Abstracts, 22:699.10, 5-HT_{2c}: Herrick-Davis, K., and Teitler, M. (1996) Society for Neuroscience Abstracts, 22:699.18; and Herrick-Davis, K. et al. (1997) J. Neurochemistry 69(3): 1138).

Small molecule modulators of serotonin receptors have been shown to have a variety of therapeutic applications such as for the treatment of any of the diseases listed above. Accordingly, there is an ongoing need for the preparation of compounds that can modulate serotonin receptors. The processes and intermediates described are directed to this and other needs.

### SUMMARY OF THE INVENTION

The present invention provides processes for preparing compounds of Formula (I):

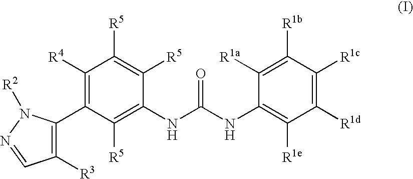

wherein constituent members are defined herein; comprising:

a) reacting a compound of Formula (II):

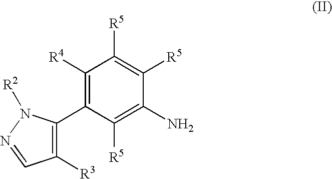

with a compound of Formula (III):

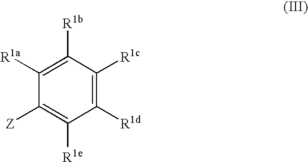

wherein Z is an isocyanate group (—NCO) or isocyanate equivalent, in a Urea Forming C₁₋₈ alcohol solvent for a time and under conditions suitable for forming said compound of Formula (I); or

b) reacting a compound of Formula (II) with an isocyanate-generating reagent for a time and under conditions suitable for forming a compound of Formula (IIa):

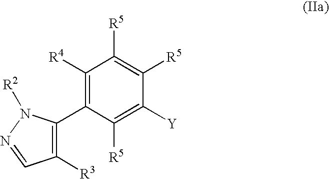

wherein Y is an isocyanate group or isocyanate equivalent; and reacting said compound of Formula (IIa) with a compound of Formula (IIIa):

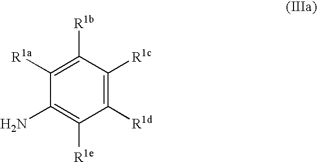

in a Urea Forming C₁₋₈ alcohol solvent for a time and under conditions suitable for forming said compound of Formula (I).

The present invention further provides processes for preparing compounds of Formula (II) comprising reacting a compound of Formula (IV):

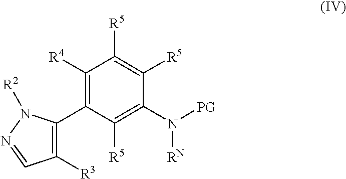

wherein:

PG is an amino protecting group; and

R^{N} is H;

or PG and R^{N} together with the N atom to which they are attached form a cyclic amino protecting group;

with an acid for a time and under conditions suitable for forming said compound of Formula (II).

The present invention further provides processes for preparing compounds of Formula (IV) comprising reacting a compound of Formula (V):

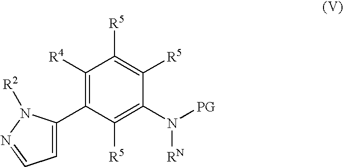

with a halogenating reagent in an amide solvent for a time and under conditions suitable for forming said compound of Formula (IV).

This application is related to U.S. Provisional Patent Application Ser. No. 60/647,613, filed Jan. 26, 2005, which is incorporated by reference in its entirety.

### BRIEF DESCRIPTION OF THE DRAWINGS

FIG. 1 shows the XRPD of the crystal form prepared by the methods of the present invention. The crystal form is referred to herein as Form II.

FIG. 2 shows the DSC of the crystal form prepared by the methods of the present invention. The crystal form is referred to herein as Form II.

### DETAILED DESCRIPTION

The present invention is directed to processes and intermediates for the preparation of substituted phenylpyrazole ureas that are useful as 5-HT_{2A} serotonin receptor modulators for the treatment of disorders mediated by 5-HT_{2A} serotonin receptor expression and/or activity such as, for example, central nervous system disorders (e.g., dementia, agitation or a symptoms thereof, behavioral disorders, psychoses, organic or NOS psychosis, drug induced psychosis, excitative psychosis, Gilles de la Tourette's syndrome, manic disorder, psychotic disorder, schizophrenia, acute schizophrenia, chronic schizophrenia, NOS schizophrenia and related disorders, and the like), cardiovascular disorders (e.g., coronary artery disease, myocardial infarction, transient ischemic attack, angina, stroke, atrial fibrillation, platelet aggregation, reducing the risk of blood clot formation, and the like), sleep disorders, asthma or symptoms thereof, diabetic-related disorders and the like.

Example processes and intermediates of the present invention are provided below in Schemes I and II, wherein constituent members of the compounds depicted therein are defined below.

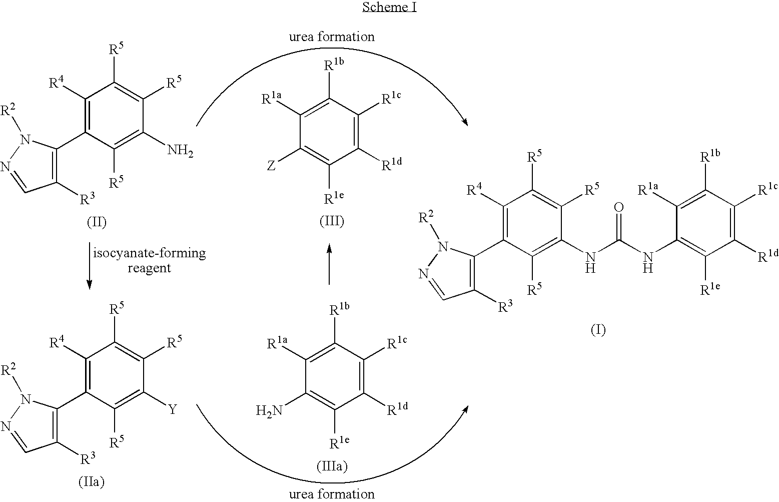

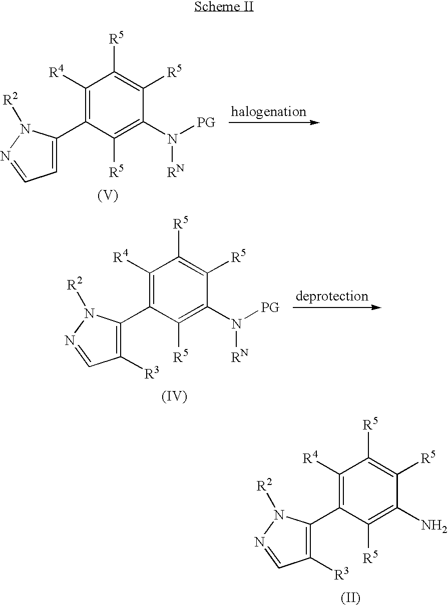

In a first aspect of the invention are provided processes, such as are exemplified by Schemes I and II (supra), that involve compounds of Formulas (I), (II), (IIa), (III), (IIIa), (IV), and (V) or salt forms thereof, wherein:

R^{1a}, R^{1b}, R^{1c}, R^{1d}, and R^{1e} are each, independently, H, halo, cyano, nitro, C₁₋₆ alkyl, C₁₋₆ haloalkyl, C₂₋₆ alkenyl, C₂₋₆ alkynyl, OR⁷, SR⁷, SOR⁸, SO₂R⁸, COR⁸, COOR⁷, OC(O)R⁸, NR⁹R¹⁰, carbocyclyl optionally substituted by one or more R⁶ or heterocyclyl optionally substituted by one or more R⁶; or R^{1a} and R^{1b}, R^{1b} and R^{1c}, R^{1c} and R^{1d}, or R^{1d} and R^{1e} together with the carbon atoms to which they are attached form a fused C₅₋₇ cycloalkyl group or fused C₅₋₇ heterocycloalkyl group; wherein each of said C₁₋₆ alkyl, C₂₋₆ alkenyl, and C₂₋₆ alkynyl, is optionally substituted with one or more C₁₋₆ acyl, C₁₋₆ acyloxy, C₁₋₆ alkoxy, C₁₋₆ thioalkoxy, carboxamide, C₁₋₆ alkylcarboxamide, C₂₋₈ dialkylcarboxamide, C₁₋₆ alkylsulfonamide, C₁₋₆ alkylsulfinyl, C₁₋₆ alkylsulfonyl, C₁₋₆ alkylureido, amino, C₁₋₆ alkylamino, C₂₋₈ dialkylamino, C₁₋₆ alkoxycarbonyl, carboxy, cyano, C₃₋₇ cycloalkyl, halogen, C₁₋₆ haloalkoxy, C₁₋₆ halothioalkoxy, C₁₋₆ haloalkyl, C₁₋₆ haloalkylsulfinyl, C₁₋₆ haloalkylsulfonyl, hydroxyl, mercapto or nitro;

R² is C₁₋₄ alkyl;

R³ is F, Cl, Br or I;

R⁴ is halo, cyano, nitro, C₁₋₆ alkyl, C₁₋₆ haloalkyl, C₂₋₆ alkenyl, C₂₋₆ alkynyl, C₁₋₆ alkoxy, SR¹¹, SOR¹², SO₂R¹², COR¹², COOR¹¹, OC(O)R¹², NR¹³R¹⁴, or C₃₋₇ cycloalkyl, wherein said C₁₋₆ alkoxy group is optionally substituted with one or more C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ thioalkoxy, C₁₋₄ alkylureido, amino, (C₁₋₆ alkoxy)carbonyl, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ halothioalkoxy, hydroxyl, nitro or phenyl optionally substituted with 1 to 5 halogen atoms;

R⁵, at each independent occurrence, is H, halo, cyano, nitro, C₁₋₆ alkyl, C₁₋₆ haloalkyl, C₂₋₆ alkenyl, C₂₋₆ alkynyl, C₁₋₆ alkoxy, SR¹¹, SOR¹², SO₂R¹², COR¹², COOR¹¹, OC(O)R¹², NR¹³R¹⁴, or C₃₋₇ cycloalkyl, wherein said C₁₋₆ alkoxy group is optionally substituted with one or more C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ thioalkoxy, C₁₋₄ alkylureido, amino, (C₁₋₆ alkoxy)carbonyl, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ halothioalkoxy, hydroxyl, nitro or phenyl optionally substituted with 1 to 5 halogen atoms;

R⁶ is halo, cyano, nitro, C₁₋₄ alkyl, C₁₋₄ haloalkyl, C₁₋₄ alkoxy, C₁₋₄ haloalkoxy, amino, (C₁₋₄ alkyl)amino, di(C₁₋₄ alkyl)amino, hydroxy, carboxy, (C₁₋₄ alkoxy)carbonyl, C₁₋₄ acyl, C₁₋₄ acyloxy, aminocarbonyl, (C₁₋₄ alkyl)aminocarbonyl, or di(C₁₋₄ alkyl)aminocarbonyl;

R⁷ and R¹¹ are each, independently, H, C₁₋₈ alkyl, C₁₋₈ haloalkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl or (5-7 membered heterocycloalkyl)alkyl;

R⁸ and R¹² are each, independently, H, C₁₋₈ alkyl, C₁₋₈ haloalkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl, (5-7 membered heterocycloalkyl)alkyl, amino, (C₁₋₄ alkyl)amino, or di(C₁₋₄ alkyl)amino;

R⁹ and R¹⁰ are each, independently, H, C₁₋₈ alkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl, (5-7 membered heterocycloalkyl)alkyl, (C₁₋₈ alkyl)carbonyl, (C₁₋₈ haloalkyl)carbonyl, (C₁₋₈ alkoxy)carbonyl, (C₁₋₈ haloalkoxy)carbonyl, (C₁₋₄ alkyl)sulfonyl, (C₁₋₄ haloalkyl)sulfonyl or arylsulfonyl;

or R⁹ and R¹⁰, together with the N atom to which they are attached form a 5-7 membered heterocycloalkyl group;

R¹³ and R¹⁴ are each, independently, H, C₁₋₈ alkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl, (5-7 membered heterocycloalkyl)alkyl, (C₁₋₈ alkyl)carbonyl, (C₁₋₈ haloalkyl)carbonyl, (C₁₋₈ alkoxy)carbonyl, (C₁₋₈ haloalkoxy)carbonyl, (C₁₋₄ alkyl)sulfonyl, (C₁₋₄ haloalkyl)sulfonyl or arylsulfonyl;

or R¹³ and R¹⁴, together with the N atom to which they are attached form a 5-7 membered heterocycloalkyl group;

PG is an amino protecting group;

R^{N} is H;

or PG and R^{N} together with the N atom to which they are attached form a cyclic amino protecting group;

R^{2a} and R^{2b} are each, independently, C₁₋₄ alkyl;

R and R′ are each, independently, C₁₋₆ alkyl, arylalkyl or alkylaryl, or R and R′ together with the O atoms to which they are attached and the intervening CH group form a 5- or 6-membered heterocycloalkyl group;

Y is an isocyanate group (—NCO) or isocyanate equivalent; and

Z is an isocyanate group (—NCO) or isocyanate equivalent.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables (e.g., R^{1a}, R^{1b}, R^{1c}, R^{1d}, R^{1e}, R², R³, R⁴, R⁵, Z, PG, R^{N}, etc.) contained within the generic chemical formulae described herein [e.g. (I), (II), (IIa), (III), (IIIa), (IV), (V), etc.] are specifically embraced by the present invention just as if they were explicitly disclosed, to the extent that such combinations embrace compounds that result in stable compounds (ie., compounds that can be isolated, characterized and tested for biological activity).

As used herein, “substituted” indicates that at least one hydrogen atom of the chemical group is replaced by a non-hydrogen substituent or group, the non-hydrogen substituent or group can be monovalent or divalent. When the substituent or group is divalent, then it is understood that this group is further substituted with another substituent or group. When a chemical group herein is “substituted” it may have up to the full valance of substitution; for example, a methyl group can be substituted by 1, 2, or 3 substituents, a methylene group can be substituted by 1 or 2 substituents, a phenyl group can be substituted by 1, 2, 3, 4, or 5 substituents, a naphthyl group can be substituted by 1, 2, 3, 4, 5, 6, or 7 substituents and the like. Likewise, “substituted with one or more substituents” refers to the substitution of a group with one substituent up to the total number of substituents physically allowed by the group. Further, when a group is substituted with more than one group, such a carbocyclyl or heterocyclyl substituted with more than one R⁶, they can be identical or they can be different.

In some embodiments, R^{1a}, R^{1b}, R^{1c}, R^{1d}, and R^{1e} are each, independently, H, halo, cyano, nitro, C₁₋₆ alkyl, C₁₋₆ haloalkyl, C₂₋₆ alkenyl, C₂₋₆ alkynyl, OR⁷, SR⁷, SOR⁸, SO₂R⁸, COR⁸, COOR⁷, OC(O)R⁸, NR⁹R¹⁰, carbocyclyl optionally substituted by one or more R⁶ or heterocyclyl optionally substituted by one or more R⁶.

It is understood that when more than one R⁶ is present they may be the same group or a different group.

In some embodiments, R^{1a}, R^{1b}, R^{1c}, R^{1d}, and R^{1e} are each, independently, H, halo, cyano, nitro, C₁₋₆ alkyl, C₁₋₆ haloalkyl, C₂₋₆ alkenyl, C₂₋₆ alkynyl, OR⁷ or carbocyclyl optionally substituted by one or more R⁶.

In some embodiments, R^{1a}, R^{1b}, R^{1c}, R^{1d}, and R^{1e} are each, independently, H, halo, C₁₋₆ alkyl, C₁₋₆ haloalkyl, C₁₋₆ alkyl, or C₁₋₆ haloalkyl.

In some embodiments, R^{1a}, R^{1b}, R^{1c}, R^{1d}, and R^{1e} are each, independently, H, F, Cl, Br, or I.

In some embodiments, R^{1a} is H or halo, R^{1b} is H, R^{1c} is halo, R^{1d} is H, and R^{1e} is H.

In some embodiments, R^{1a} is halo, R^{1b} is H, R^{1c} is halo, R^{1d} is H, and R^{1e} is H.

In some embodiments:

R^{1a} is F, R^{1b} is H, R^{1c} is F, R^{1d} is H, and R^{1e} is H;

R^{1a} is H, R^{1b} is H, R^{1c} is Cl, R^{1d} is H, and R^{1e} is H;

R^{1a} is H, R^{1b} is H, R^{1c} is F, R^{1d} is H, and R^{1e} G is H; or

R^{1a} is H, R^{1b} is H, R^{1c} is Cl, R^{1d} is H, and R^{1e} is H.

In some embodiments, R² is methyl or ethyl.

In some embodiments, R² is methyl.

In some embodiments, R³ is Cl or Br.

In some embodiments, R³ is Br.

In some embodiments, R⁴ is halo, cyano, nitro, C₁₋₆ alkyl, C₁₋₆ haloalkyl, C₂₋₆ alkenyl, C₂₋₆ alkynyl, C₁₋₆ alkoxy, wherein said C₁₋₆ alkoxy group is optionally substituted with one or more C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ thioalkoxy, C₁₋₄ alkylureido, amino, (C₁₋₆ alkoxy)carbonyl, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ halothioalkoxy, hydroxyl, nitro or phenyl optionally substituted with 1 to 5 halogen atoms.

In some embodiments, R⁴ is C₁₋₆ alkoxy optionally substituted with one or more C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ thioalkoxy, C₁₋₄ alkylureido, amino, (C₁₋₆ alkoxy)carbonyl, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ halothioalkoxy, hydroxyl, nitro or phenyl optionally substituted with 1 to 5 halogen atoms.

In some embodiments, R⁴ is C₁₋₆ alkoxy.

In some embodiments, R⁴ is C₁₋₃ alkoxy.

In some embodiments, R⁴ is methoxy or ethoxy.

In some embodiments, R⁴ is methoxy.

In some embodiments, R⁵, at each independent occurrence, is H, halo, cyano, nitro, C₁₋₆ alkyl, C₁₋₆ haloalkyl, C₂₋₆ alkenyl, C₂₋₆ alkynyl, or C₁₋₆ alkoxy.

In some embodiments, R⁵, at each independent occurrence, is H or halo.

In some embodiments, R⁵, at each occurrence, is H.

In some embodiments, R and R′ are both C₁₋₄ alkyl.

In some embodiments, R and R′ are both methyl.

In some embodiments, R^{2a} and R^{2b} are both methyl.

In some embodiments, PG is an acyl group.

In some embodiments, PG is —C(O)—(C₁₋₄ alkyl).

In some embodiments, PG is —C(O)Me.

In some embodiments:

R^{1a}, R^{1b}, R^{1c}, R^{1d}, and R^{1e} are each, independently, H, halo, cyano, nitro, C₁₋₆ alkyl, C₁₋₆ haloalkyl, C₂₋₆ alkenyl, C₂₋₆ alkynyl, OR⁷, SR⁷, SOR⁸, SO₂R⁸, COR⁸, COOR⁷, OC(O)R⁸, NR⁹R¹⁰, carbocyclyl optionally substituted by one or more R⁶ or heterocyclyl optionally substituted by one or more R⁶;

R³ is F, Cl, Br or I;

R⁴ is halo, cyano, nitro, C₁₋₆ alkyl, C₁₋₆ haloalkyl, C₂₋₆ alkenyl, C₂₋₆ alkynyl, C₁₋₆ alkoxy, wherein said C₁₋₆ alkoxy group is optionally substituted with one or more C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, Cl₄ thioalkoxy, C₁₋₄ alkylureido, amino, (C₁₋₆ alkoxy)carbonyl, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ halothioalkoxy, hydroxyl, nitro or phenyl optionally substituted with 1 to 5 halogen, atoms; and

R⁵, at each independent occurrence, is H, halo, cyano, nitro, C₁₋₆ alkyl, C₁₋₆ haloalkyl, C₂₋₆ alkenyl, C₂₋₆ alkynyl, or C₁₋₆ alkoxy.

In some embodiments:

R^{1a}, R^{1b}, R^{1c}, R^{1d}, and R^{1e} are each, independently, H, halo, C₁₋₆ alkyl, C₁₋₆ haloalkyl, C₁₋₆ alkyl, or C₁₋₆ haloalkyl;

R³ is F, Cl, Br or I;

R⁴ is C₁₋₆ alkoxy group optionally substituted with one or more C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ thioalkoxy, C₁₋₄ alkylureido, amino, (C₁₋₆ alkoxy)carbonyl, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ halothioalkoxy, hydroxyl, nitro or phenyl optionally substituted with 1 to 5 halogen atoms; and

R⁵, at each occurrence, is H.

In some embodiments:
R^{1a}, R^{1b}, R^{1c}, R^{1d}, and R^{1e} are each, independently, H, F, Cl, Br or I;
R² is methyl or ethyl;
R³ is F, Cl, Br or I;
R⁴ is C₁₋₆ alkoxy; and
R⁵, at each occurrence, is H.

In some embodiments:
R^{1a}, R^{1b}, R^{1c}, R^{1d}, and R^{1e} are each, independently, H, F, or Cl;
R² is methyl;
R³ is Cl or Br;
R⁴ is methoxy; and
R⁵, at each occurrence, is H.

In some embodiments:
R^{1a} is F;
R^{1b} is H;
R^{1c} is F;
R^{1d} is H;
R^{1e} is H;
R² is methyl;
R³ is Br;
R⁴ is methoxy; and
R⁵, at each occurrence, is H.

In some embodiments:
R^{1a} is H;
R^{1b} is H;
R^{1c} is Cl;
R^{1d} is H;
R^{1e} is H;
R² is methyl;
R³ is Br;
R⁴ is methoxy; and
R⁵, at each occurrence, is H.

In some embodiments:
R^{1a} is H;
R^{1b} is H;
R^{1c} is F;
R^{1d} is H;
R^{1e} is H;
R² is methyl;
R³ is Br;
R⁴ is methoxy; and
R⁵, at each occurrence, is H.

In some embodiments:
R^{1a} is H;
R^{1b} is H;
R^{1c} is Cl;
R^{1d} is H;
R^{1e} is H;
R² is methyl;
R³ is Cl;
R⁴ is methoxy; and
R⁵, at each occurrence, is H.

In some embodiments, Z is —NCO.

In some embodiments, Y is —NCO.

In some embodiments:

R³ is F, Cl, Br or I;

R⁴ is halo, cyano, nitro, C₁₋₆ alkyl, C₁₋₆ haloalkyl, C₂₋₆ alkenyl, C₂₋₆ alkynyl, C₁₋₆ alkoxy, wherein said C₁₋₆ alkoxy group is optionally substituted with one or more C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, Cl₄ alkylsulfonamide, C₁₋₄ alkylsulfinyl, C₁₋₄ alkylsulfonyl, C₁₋₄ thioalkoxy, C₁₋₄ alkylureido, amino, (C₁₋₆ alkoxy)carbonyl, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ halothioalkoxy, hydroxyl, nitro or phenyl optionally substituted with 1 to 5 halogen atoms; and

R⁵ is H, halo, cyano, nitro, C₁₋₆ alkyl, C₁₋₆ haloalkyl, C₂₋₆ alkenyl, C₂₋₆ alkynyl, or C₁₋₆ alkoxy.

In some embodiments:

R³ is F, Cl, Br or I;

R⁴ is C₁₋₆ alkoxy group optionally substituted with one or more C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ thioalkoxy, C₁₋₄ alkylureido, amino, (C₁₋₆ alkoxy)carbonyl, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ halothioalkoxy, hydroxyl, nitro or phenyl optionally substituted with 1 to 5 halogen atoms; and

R⁵, at each occurrence, is H.

In some embodiments:
R² is methyl or ethyl;
R³ is F, Cl, Br or I;
R⁴ is C₁₋₆ alkoxy; and
R⁵, at each occurrence, is H.

In some embodiments:
R² is methyl;
R³ is Cl or Br;
R⁴ is methoxy; and
R⁵, at each occurrence, is H.

In some embodiments, for compounds of Formula (II), R² is methyl; R³ is Cl or Br; R⁴ is methoxy; and R⁵, at each occurrence, is H.

In some embodiments, for compounds of Formula (II), R² is methyl; R³ is Br; R⁴ is methoxy; and R⁵, at each occurrence, is H.

In some embodiments, for compounds of Formula (II), R² is methyl; R³ is Cl; R⁴ is methoxy; and R⁵, at each occurrence, is H.

In some embodiments, for compounds of Formula (IV), R² is methyl; R³ is Br; R⁴ is methoxy; R⁵, at each occurrence, is H; and PG is —C(O)Me.

In some embodiments, for compounds of Formula (IV), R² is methyl; R³ is Cl; R⁴ is methoxy; R⁵, at each occurrence, is H; and PG is —C(O)Me.

In some embodiments, for compounds of Formula (V), R² is methyl; R⁴ is methoxy; R⁵, at each occurrence, is H; and PG is —C(O)Me.

In some embodiments, for compounds of Formula (VI), R^{2a} is methyl; R^{2b} is methyl; R⁴ is methoxy; R⁵, at each occurrence, is H; and PG is C(O)Me.

### Urea Forming Step

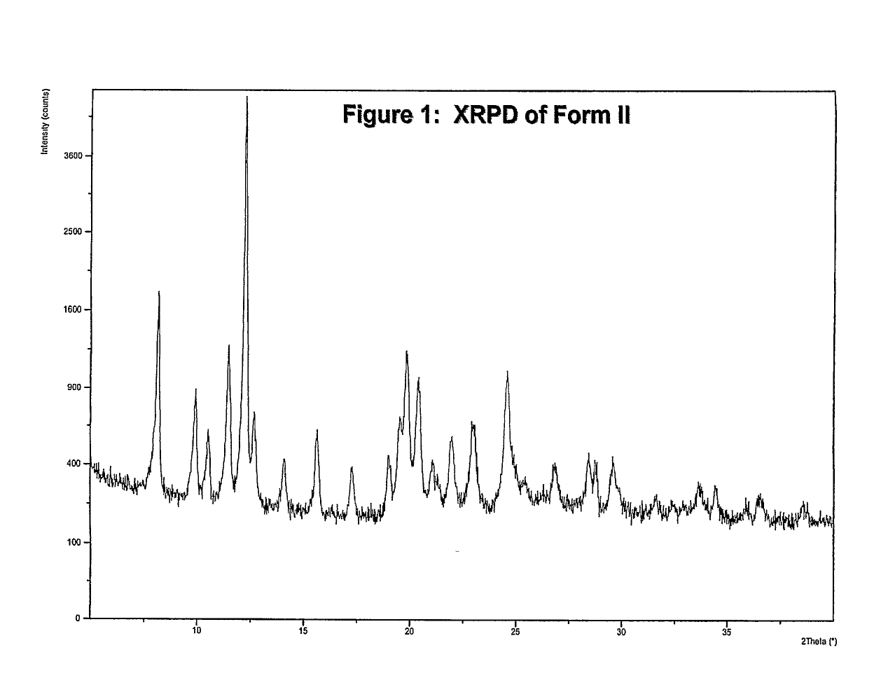

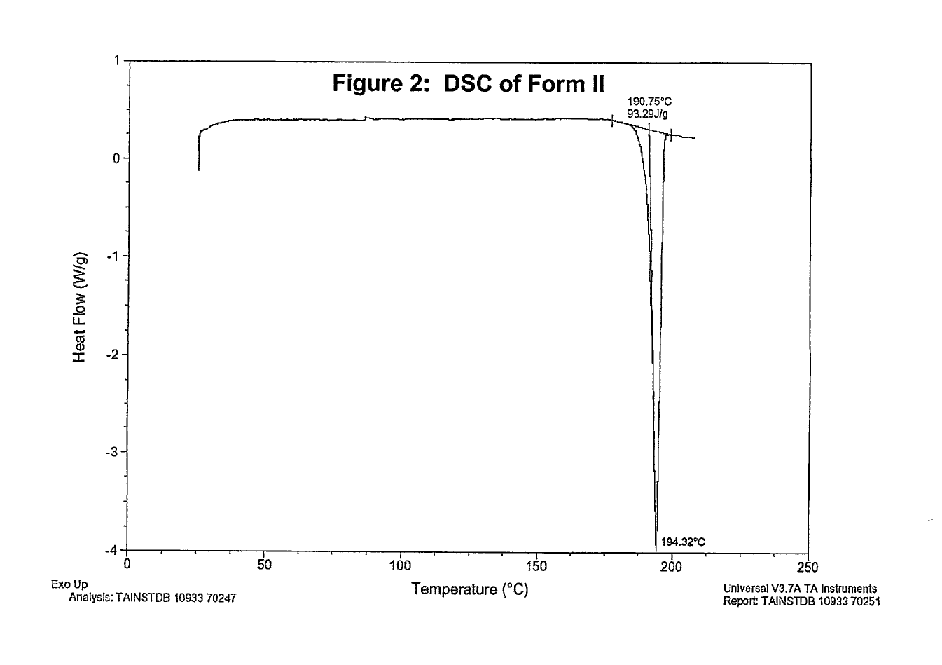

The chemical reactions resulting in compounds of Formula (I) and formation of the urea linkage can be carried out by any of the numerous methods known in the art. Surprisingly however, it was discovered that the Urea Forming Step can be conducted using an alcohol as a solvent, referred herein as “Urea Forming C₁₋₈ alcohol solvent.” The use of an alcohol solvent in the Urea Forming Step not only provides significant cost advantages but also produces a highly desirable crystal form that has formulation benefits and enhanced stability (XRPD and DSC provided in FIGS. 1 and 2 respectively). In addition, cost saving also results from the ability to telescope backwards to the bromination step. Therefore, the Halogenation, Deprotection and Urea Forming Stepscan all be performed in the same solvent without isolation of intermediates.

Examples of Urea Forming processes according to the present invention are depicted in Schemes I and II. Accordingly, the compound of Formula (I):

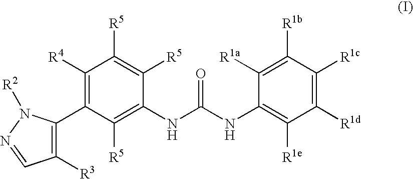

wherein constituent members are defined herein, can be prepared by:

reacting a compound of Formula (II):

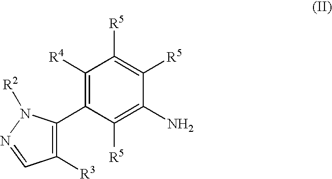

with a compound of Formula (II):

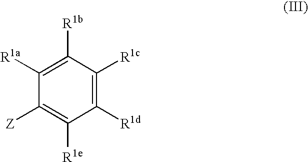

wherein Z is an isocyanate group (—NCO) or isocyanate equivalent, in a Urea Forming C₁₋₈ alcohol solvent for a time and under conditions suitable for forming said compound of Formula (I); or

b) reacting a compound of Formula (II) with an isocyanate-generating reagent for a time and under conditions suitable for forming a compound of Formula (IIa):

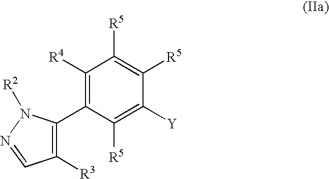

wherein Y is an isocyanate group or isocyanate equivalent; and reacting said compound of Formula (IIa) with a compound of Formula (IIIa):

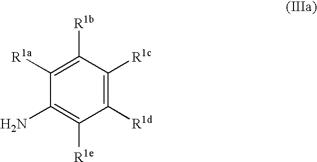

in a Urea Forming C₁₋₈ alcohol solvent for a time and under conditions suitable for forming said compound of Formula (I).

In some embodiments, the reactants are of Formulae (II) and (III) wherein Z is an isocyanate group, in a Urea Forming C₁₋₈ alcohol solvent for a time and under conditions suitable for forming said compound of Formula (I).

The Urea Forming Step is carried out in a solvent comprising a Urea Forming C₁₋₈ alcohol solvent.

In some embodiments, the Urea Forming C₁₋₈ alcohol solvent comprises a 1° alcohol or 2° alcohol.

In some embodiments, the Urea Forming C₁₋₈ alcohol solvent comprises a 1° alcohol. In some embodiments, the 1° alcohol is selected from the group consisting of methanol, ethanol, 1-propanol, 1-butanol and 2-methyl-propan-1-ol. In some embodiments, the 1° alcohol is methanol. In some embodiments, the 1° alcohol is 1-propanol.

In some embodiments, the Urea Forming C₁₋₈ alcohol solvent comprises a 2° alcohol. In some embodiments, the 2° alcohol is 2-propanol.

The urea-forming reaction can be carried out at any temperature. For example, suitable temperatures include those less than about 90° C. In some embodiments, suitable temperatures include those less than about 75° C. In some embodiments, the reaction is carried out at temperatures between about 5° C. to about 90° C. In some embodiments, the reaction is carried out at temperatures between about 25° C. to about 75° C. In some embodiments, the reaction is carried out at temperatures between about 30° C. to about 60° C. In some embodiments, the reaction is carried out at temperatures between about 40° C. to about 50° C. In some embodiments, the reaction is carried out at temperatures between about −5° C. to about 75° C. In some embodiments, the reaction is carried out at temperatures between about 15° C. to about 60° C.

In some embodiments, the reaction is carried out under an inert atmosphere.

In some embodiments, the reaction is carried out wherein the compound of Formula (III) is added to a solution containing said compound of Formula (II).

In some embodiments, the reaction is carried out wherein the compound of Formula (III) is added portionwise to a solution containing the compound of Formula (II). It is understood that portionwise encompasses any method where a compound of Formula (III) is added other than all at once, examples include, addition of a neat solution or solid, addition of a solution containing the compound of Formula (III), and the like.

In some embodiments, the reaction is carried out wherein the compound of Formula (II) is added to a solution containing the compound of Formula (III). In some embodiments, the addition is carried out portionwise, either as a solid or a solution wherein the compound of Formula (II) is dissolved in the Urea Forming C₁₋₈ alcohol solvent prior to addition.

In some embodiments, the reactants bearing the isocyanate or isocyanate equivalent groups (e.g., compounds (III)) are provided in equal amounts relative to the amount of aniline (e.g., compounds of Formula (II)). In some embodiments, the compound of Formula (III) is added in molar excess relative to the amount of Formula (II). For example, the molar ratio of a compound of Formula (III) to a compound of Formula (II) can be about 1:1 to about 1.5:1 or about 1:1 to about 1.2:1.

In some embodiments, after the addition of the compound of Formula (III) the temperature is increased to a temperature of the boiling point of the reaction mixture. In some embodiments, after the addition of the compound of Formula (III) the temperature is increased to between about 35° C. to about 100° C. In some embodiments, after the addition of the Compound (III) the temperature is increased to between about 45° C. to about 70° C. In some embodiments, after the addition of the Compound (III) the temperature is increased to between about 60° C. to about 100° C. In some embodiments, after the addition of the Compound (III) the temperature is increased to between about 70° C. to about 90° C.

In some embodiments, the aniline starting material (e.g., a compound of Formula (II)) can be dissolved in the Urea Forming C₁₋₈ alcohol solvent prior to the reaction, thus forming a solution.

In some embodiments, the compound of Formula (I) is prepared by reacting a compound of Formula (II) with a compound of Formula (III). In alternate embodiments, the compound of Formula (I) is prepared by reacting a compound of Formula (IIa) with a compound of Formula (IIIa).

Starting materials bearing isocyanate and isocyanate equivalent moieties are well known in the art and commercially available. These can also be routinely prepared from corresponding anilines by reaction with an isocyanate-generating reagent, which includes materials that react with the amino group of an aniline to form an isocyanate equivalent group. For example, an isocyanate-bearing compound can be readily prepared by reacting the corresponding aniline with an isocyanate-generating reagent such as, for example, phosgene (i.e., Cl₂C═O) or triphosgene [i.e., bis-trichloromethyl carbonate, Cl₃COC(O)OCCl₃] to generate the isocyanate derivative which can then be optionally isolated. Another procedure for preparing isocyanates involves using the isocyanate-generating reagent di-t-butyltricarbonate to generate isocyanates from anilines in a similar manner as described above. An example of this procedure is reported by Peerlings et al. in Tetrahedron Lett. 1999, 40, 1021-1024, the disclosure of which is incorporated herein by reference in its entirety. These procedures and others known in the art can give rise to isocyanates as illustrated in Schemes III and IV below.

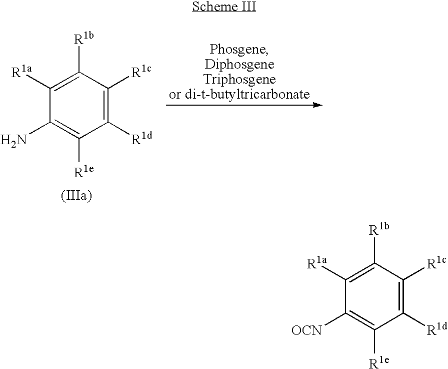

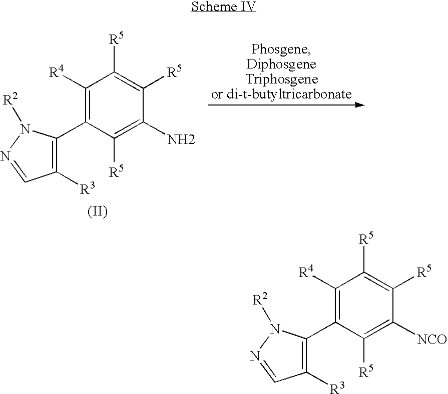

An isocyanate equivalent includes a moiety other than isocyanate that is able to form a urea linkage upon reaction with an aniline (e.g., compounds of Formulae (II)). Isocyanate equivalents can be prepared from the corresponding anilines by the sequential action of the isocyanate-generating reagents: 1) carbonyl diimidazole and 2) methyl iodide in THF and acetonitrile, respectively, as described, for example, by Batey et al. in Tetrahedron Lett. 1998, 39, 6267-6270, the disclosure of which is incorporated herein by reference in its entirety. This procedure can give rise to isocyanate equivalents as illustrated in Schemes V and VI below.

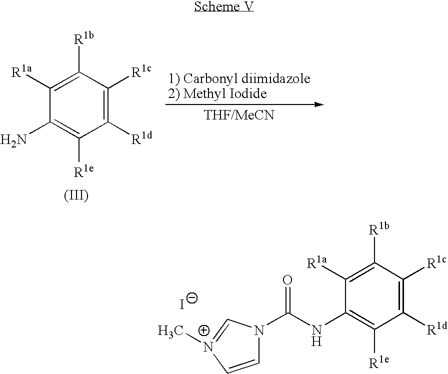

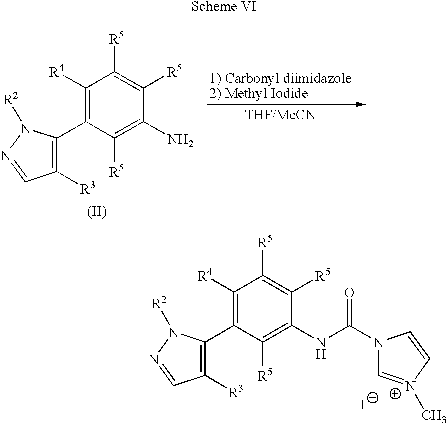

Other isocyanate equivalents can be generated by reacting the corresponding aniline with an isocyanate-generating reagent such a substituted alkyl chloroformate of Formula:

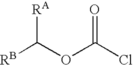

wherein R^{A} is C₁₋₈ alkyl and R^{B} is a leaving group, for a time and under conditions suitable for forming the isocyanate equivalent. In some embodiments, R^{A} is methyl. In further embodiments, R^{B} is Cl, Br, I, mesylate, tosylate or the like. In still further embodiments, R^{B} is Cl, Br or I; and in yet further embodiments, R^{B} is Cl.

Formation of isocyanate equivalents using a substituted alkyl chloroformate is illustrated in Schemes VII and VIII below.

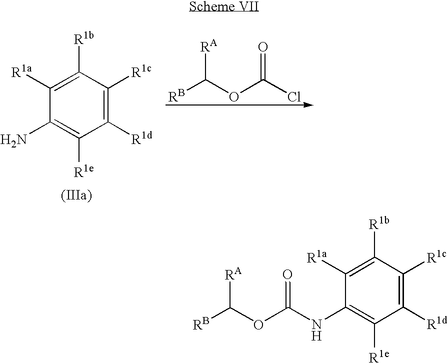

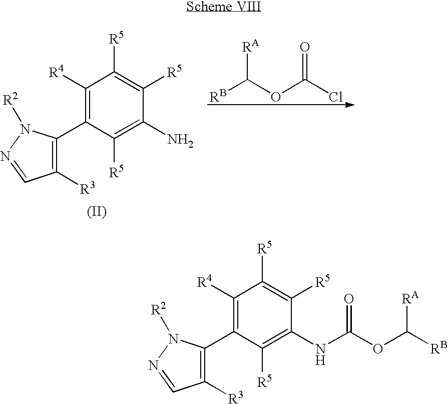

Reaction of anilines (e.g., compounds of Formula (II) and (IIIa)) such as those described in Schemes VII and VIII with the isocyanate-generating reagent substituted alkylchloroformate can be optionally carried out in the presence of an organic base. Suitable organic bases include, for example, pyridine, dimethylaminopyridine, piperidine, morpholine, mixtures thereof and the like. In some embodiments, the organic base is pyridine. The organic base can, in some instances, replace the leaving group R^{B} to form an organic base derivative. In some embodiments, pyridine replaces the leaving group R^{B} to form a pyridinium derivative.

Generally, the molar ratio of an aniline, such as a compound of Formula (II) or (IIIa), to a substituted alkylchloroformate can range from about 1:1 to about 1:2. In some embodiments, the ratio is about 1:1 to about 1:1.5. Such reactions can be carried out at any suitable temperature such as, for example, about 0 to about 60° C. or about 10 to about 45° C.

It is generally understood that although the isocyanate or isocyanate equivalent can be isolated, it can also be generated in situ and used directly to complete the urea formation reaction. Accordingly, in some embodiments, the isocyanate or isocyanate equivalent is generated in situ and reacted directly with the appropriate aniline without isolation.

### Deprotection

According to a further aspect of the invention, a compound of Formula (II) can be prepared by the process comprising reacting a compound of Formula (V):

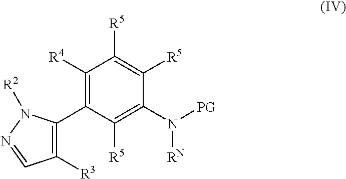

wherein:

PG is an amino protecting group; and

R^{N} is H; or PG and R^{N} together with the N atom to which they are attached form a cyclic amino protecting group;

with an acid for a time and under conditions suitable for forming said compound of Formula (a).

In some embodiments, PG is an acyl group.

In some embodiments, PG is —C(O)—(C₁₋₆ alkyl).

In some embodiments, PG is —C(O)Me.

While numerous suitable deprotecting agents are known that can selectively remove an amino protecting group it was discovered that the deprotection of the amino group in the present invention can be advantageously conducted in the presence of an acid. This is contrary to what was reported in International Publication Number WO 2004/028450 wherein a time course for a deprotection of an amino group in the presence of an acid resulted in lost of the bromine at the C(4) position of the pyrazole, specifically forming 1.7%, 6.3% and 22.8% of the des-bromo compound at 1, 6 and 21 hours respectively. It was discovered that the amino protecting group can be cleanly and efficiently removed with an acid reagent without loss or scrambling of the bromine substituent.

The chemistry of protecting groups that use an acid reagent for deprotections can be found, for example, in Green and Wuts, Protective Groups in Organic Synthesis, 3rd. Ed., Wiley & Sons, 1999, which is incorporated herein by reference in its entirety.

The deprotection can be carried with an acid.

In some embodiments, the molar ratio of acid to compound of Formula (IV) is greater than about 1. In some embodiments, the molar ratio of acid to compound of Formula (IV) is between about 1 to about 8. In some embodiments, the molar ratio of acid to compound of Formula (IV) is between about 2 to about 4.

In some embodiments, the acid is selected from the group consisting of HCl, HBr, sulfuric acid, methane sulfonic acid, trifluoromethane sulfonic acid and p-toluene sulfonic acid.

In some embodiments, the acid comprises sulfuric acid.

In some embodiments, the acid comprises HCl. It is understood that HCl can be introduced via a variety of methods, for example, HCl can be bubbled into the reaction as a gas, HCl can be added as a solution, and the like. In some embodiments, the HCl is generated in situ via reaction of an acyl halide and said Deprotecting C₁₋₈ alcohol solvent. In some embodiments, the acyl halide is (C₁₋₆ alkyl)-C(O)—Cl. In some embodiments, the acyl halide is Me-C(O)—Cl (i.e., acetyl chloride). In some embodiments, the Deprotecting C₁₋₈ alcohol solvent is a 1° alcohol. In some embodiments, the Deprotecting C₁₋₈ alcohol solvent is selected from the group consisting of methanol, ethanol, 1-propanol and 1-butanol. In some embodiments, the Deprotecting C₁₋₈ alcohol solvent is methanol. In some embodiments, the Deprotecting C₁₋₈ alcohol solvent is 1-propanol. In some embodiments, the HCl is generated under essentially anhydrous conditions. In some embodiments, the molar ratio of HCl to compound of Formula (IV) is greater than about 1. In some embodiments, the molar ratio of HCl to compound of Formula (IV) is between about 2 to about 4.

The deprotection can be optionally carried out in an organic solvent.

In some embodiments, the organic solvent comprises a Deprotecting C₁₋₈ alcohol solvent.

In some embodiments, the Deprotecting C₁₋₈ alcohol solvent comprises a 1° alcohol or 2° alcohol.

In some embodiments, the Deprotecting C₁₋₈ alcohol solvent comprises a 1° alcohol. In some embodiments, the 1° alcohol is selected from the group consisting of methanol, ethanol, 1-propanol, 1-butanol and 2-methyl-propan-1-ol. In some embodiments, the 1° alcohol is methanol. In some embodiments, the 1° alcohol is 1-propanol.

In some embodiments, the Deprotecting C₁₋₈ alcohol solvent comprises a 2° alcohol. In some embodiments, the 2° alcohol is 2-propanol.

The deprotection can be carried out at any suitable temperature. In some embodiments, the deprotection is carried out at a temperature above about 20° C. In some embodiments, the deprotection is carried out at a temperature between about 20° C. to about 120° C. In some embodiments, the deprotection is carried out at a temperature between about 55° C. to about 100° C. In some embodiments, the deprotection is carried out at reflux temperature.

In some embodiments, the deprotection step results in formation of less than about 2 mole % of a compound of Formula (IIb):

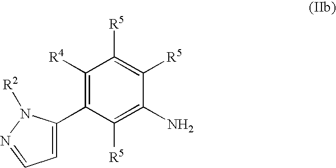

relative to the amount of compound of Formula (II).

In some embodiments, the deprotection step results in formation of less than about 1 mole % of a compound of Formula (IIb).

In some embodiments, the deprotection step results in formation of less than about 0.5 mole % of a compound of Formula (IIb).

In some embodiments, the deprotection step results formation of an essentially undetectable amount of a compound of Formula (IIb).

Methods that can be used to determine relative amounts of compounds in a sample or monitor reactions are readily credited to those skilled in the art; HPLC is one method that is commonly used. A variety of detection methods can be used in connection with an HPLC, such as UV, MS, diode-array, and the like. One representative set of conditions is provided here:

Instrument: Waters 2695

Column: Waters SymmetryShield RP18, 3.5 μm, 4.6×150 mm or equivalent with pre-column filter;

Mobile Phase A: Deionized Water

Mobile Phase B: Acetonitrile

Needle Rinse Acetonitrile

Flow Rate: 1.5 mL/min.

Column Temperature: 45° C.

Detector Wavelength: 252 nm

Sample Injection Volume: 10 μL

Gradient Profile

| Time (min) | Flow rate mL/min | % A | % B | Curve |
|---|---|---|---|---|
| 0 | 1.5 | 80 | 20 | — |
| 30 | 1.5 | 33 | 67 | 6 |
| 32 | 1.5 | 80 | 20 | 1 |

Data acquisition time: 30 minutes; Gradient re-equilibration time: 2 minutes

In some embodiments, the compound of Formula (II) is not physically isolated but carried on directly into the Urea Forming Step thus combining or “telescoping” the Deprotection and Urea Forming Steps. Accordingly, in some embodiments, the Deprotecting C₁₋₈ alcohol solvent is essentially the same as the Urea Forming C₁₋₈ alcohol solvent. In some embodiments, the Deprotecting C₁₋₈ alcohol solvent and Urea forming C₁₋₈ alcohol solvent both comprise 1-propanol. In other words, the Deprotecting C₁₋₈ alcohol solvent used in preparing compound (II) is essentially the same solvent as the Urea forming C₁₋₈ alcohol solvent in the Urea Forming Step.

Alternatively, the Deprotection and Urea Forming Steps can be conducted using the essentially the same solvent but the deprotection is carried out under basic conditions. Accordingly, one aspect of the present invention includes combining or “telescoping” the Deprotection and Urea Forming Steps wherein the deprotection step is carried out under basic conditions.

In some embodiments, the compound of Formula (II) is prepared by the process comprising reacting a compound of Formula (IV):

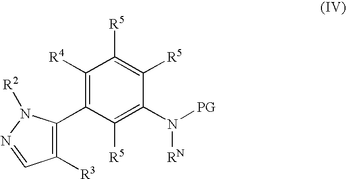

wherein:

PG is an amino protecting group; and

R^{N} is H;

or PG and R^{N} together with the N atom to which they are attached form a cyclic amino protecting group;

with a base for a time and under conditions suitable for forming said compound of Formula (II).

In some embodiments, PG is an acyl group.

In some embodiments, PG is —C(O)—(C₁₋₆ alkyl).

In some embodiments, PG is —C(O)Me.

In some embodiments, the base is sodium hydroxide.

In some embodiments, the reaction is carried out in an organic solvent.

In some embodiments, the organic solvent comprises a Deprotecting C₁₋₈ alcohol solvent.

In some embodiments, the Deprotecting C₁₋₈ alcohol solvent comprises a 1° alcohol or 2° alcohol.

In some embodiments, the Deprotecting C₁₋₈ alcohol solvent comprises a 1° alcohol.

In some embodiments, the 1° alcohol is selected from the group consisting of methanol, ethanol, 1-propanol, 1-butanol and 2-methyl-propan-1-ol. In some embodiments, the 1° alcohol is methanol. In some embodiments, the 1° alcohol is 1-propanol.

In some embodiments, the Deprotecting C₁₋₈ alcohol solvent comprises a 2° alcohol. In some embodiments, the 2° alcohol is 2-propanol.

Deprotection under basic conditions can be conducted at any suitable temperature. In some embodiments, deprotection is carried out at a temperature above about 20° C. In some embodiments, deprotection is carried out at a temperature between about 20° C. to about 120° C. In some embodiments, deprotection is carried out at a temperature between about 70 to about 90° C. In some embodiments, deprotection is carried out at a temperature between about 55° C. to about 100° C. In some embodiments, deprotection is carried out at reflux temperature.

In some embodiments, deprotection is carried out under an inert atmosphere.

In some embodiments, deprotection is carried out under a N₂ atmosphere.

In some embodiments, deprotection results in less than about 3 mole % of a compound of Formula (IIb):

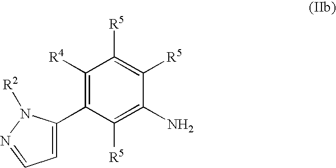

relative to the amount of compound of Formula (II).

In some embodiments, deprotection comprises less than about 1 mole % of a compound of Formula (IIb).

In some embodiments, deprotection results in essentially an undetectable amount of

### Halogenation

In further aspects of the invention, a compound of Formula (IV) is prepared by the process comprising reacting a compound of Formula (V):

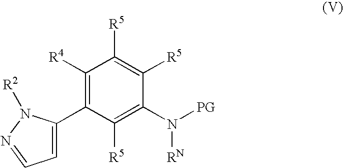

with a halogenating reagent in an amide solvent for a time and under conditions suitable for forming said compound of Formula (IV).

Any of the numerous halogenating reagents known in the art can be used. In some embodiments, the halogenating reagent is a brominating or chlorinating reagent. Some example brominating reagents include, for example, Br₂, N-bromosuccinimide (NBS), 1,3-dibromo-5,5 dimethylhydantoin, pyridinium tribromide (pyrHBr₃) and the like. An example chlorinating reagent is N-chlorosuccinimide. In some embodiments, the halogenating reagent is N-bromosuccinimide.

Although any number of the halogenating reagents known in the art can be used in the Halogenating Step it was discovered that an amide solvent is needed to provide clean conversion of a compound of Formula (V) to a compound of Formula (IV) that can be subsequently isolated substantially free of the compound of Formula (V).

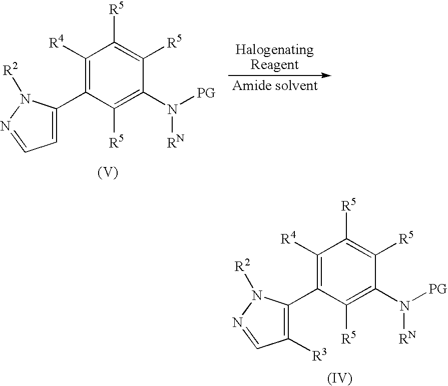

The use of DMF, methanol and ethanol in the Halogenating Step provides about 3 to 4% of the starting material (e.g., compound of Formula (V)) upon isolation. Although the halogenation under these conditions proceeds to completion (as determined via HPLC), surprisingly, it was observed that during isolation of the product (e.g., compound of Formula (IV)) about 3 to 4% of the starting material (e.g., compound of Formula (V)) was routinely obtained. This amount of compound, which is now an impurity in subsequent steps, can undergo similar reactions as the desired compounds and as a result is very difficult to remove in subsequent steps. In general, removal of this impurity required recrystallization(s) and associated yield loss to obtain levels of about 1%.

Inventors made the critical discovery that replacing the above mentioned problem solvents with an amide solvent allowed the compounds of Formula (IV) to be isolated cleanly. Carrying out the reaction in this solvent provided the isolated product without contamination and eliminated the need for recrystallization(s) and loss of material related thereto.

The halogenating reaction can be conducted using any suitable amide solvent. As used herein an amide solvent has the Formula:

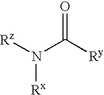

wherein R^{z} and R^{x} are each independently H or C₁₋₄ alkyl and R^{y} is C₁₋₄ alkyl; or R^{x} and R^{y} together with the amide group form a 5 or 6 membered lactam represented by the two formulae:

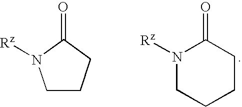

In some embodiments, the amide solvent in the halogenating reaction is dimethylacetamide or N-methyl-2-pyrrolidone. In some embodiments, the amide solvent in the halogenating reaction is dimethylacetamide.

The halogenating reaction can be conducted at any suitable temperature. In some embodiments, the reaction is carried out at a temperature about 70° C. or below. In some embodiments, the reaction is carried out at a temperature about 50° C. or below. In some embodiments, the reaction is carried out at a temperature about 30° C. or below. In some embodiments, the reaction is carried out at a temperature about 25° C. or below. In some embodiments, the reaction is carried out at a temperature about 25° C. to about 0° C.

In some embodiments, the halogenating reaction results in about 98 mol % conversion or higher of the compound of Formula (IV) compared to the compound of Formula (V) and isolated the compound of Formula (IV) containing about 2 mol % or lower of the compound of Formula (V).

In some embodiments, the halogenating reaction results in about 99 mole % conversion or higher of the compound of Formula (IV) compared to the compound of Formula (V) and isolated the compound of Formula (IV) containing about 1 mol % or lower of the compound of Formula (V).

In some embodiments, the halogenating reaction results in essentially an undetectable amount of the compound of Formula (V) compared to the compound of Formula (V) and isolated the compound of Formula (IV) essentially free of the compound of Formula (V).

Methods that can be used to determine relative amounts of compounds in a sample or to monitor reactions are readily credited to those skilled in the art; HPLC is just one method that is commonly used. A variety of detection methods can be used in connection with an HPLC, such as UV, MS, diode-array, and the like. The mol % used herein can be determined by HPLC with a UV detector. One representative set of conditions is provided supra.

Isolation of the compounds of Formula (IV) in the presence of dilute acid, such as HCl and the like assists in minimizing dehalogenation. Accordingly, in some embodiments, isolation of the compounds of Formula (IV) is carried out in the presence of dilute acid. In some embodiments, the diluted acid is aqueous HCl. In some embodiments, the dilute acid is about 0.1M to about 1.0M aqueous HCl. In some embodiments, the dilute acid is about 0.4M to about 0.8M aqueous HCl.

In further aspects of the invention, a compound of Formula (IV) is prepared by the process comprising reacting a compound of Formula (V):

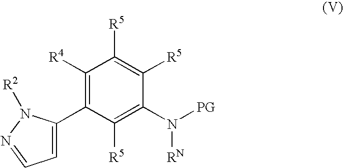

with a Halogenating reagent, in an amide solvent, a Halogenating C₁₋₈ alcohol solvent or mixture thereof, for a time and under conditions suitable for forming said compound of Formula (IV); wherein the Halogenating reagent and amide solvent are as described supra.

It was discovered that compounds of Formula (IV) can be prepared and used directly in the Deprotection Step without difficulties in purity as described supra. Accordingly, in some embodiments, the Halogenating C₁₋₈ alcohol solvent and the Deprotecting C₁₋₈ alcohol alcohol solvent are essentially same. In some embodiments, a compound of Formula (V) can be halogenated, deprotected and converted to a urea (e.g., compound of Formula (I)) in the same alcohol solvent. In some embodiments, the Urea forming C₁₋₈ alcohol solvent, Deprotecting C₁₋₈ alcohol solvent and the Halogenating C₁₋₈ alcohol solvent can be essentially the same.

In some embodiments, the Halogenating C₁₋₈ alcohol solvent comprises a 1° alcohol or 2° alcohol.

In some embodiments, the Halogenating C₁₋₈ alcohol solvent comprises a 1° alcohol. In some embodiments, the 1° alcohol is selected from the group consisting of methanol, ethanol, 1-propanol, 1-butanol and 2-methyl-propan-1-ol. In some embodiments, the 1° alcohol is 1-propanol.

In some embodiments, the Halogenating C₁₋₈ alcohol solvent comprises a 2° alcohol. In some embodiments, the 2° alcohol is 2-propanol.

In some embodiments, the Halogenating C₁₋₈ alcohol solvent is essentially the same as said Urea Forming C₁₋₈ alcohol solvent and said Deprotecting C₁₋₈ alcohol solvent.

In some embodiments, the Urea forming C₁₋₈ alcohol solvent and said Deprotecting C₁₋₈ alcohol solvent and said Halogenating C₁₋₈ alcohol solvent all comprise 1-propanol.

In some embodiments, the compound of Formula (IV) is not isolated.

In some embodiments, the compound of Formula (II) is not isolated.

### DEFINITIONS

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms.

As used herein, “alkenyl” refers to an alkyl group having one or more double carbon-carbon bonds. Example alkenyl groups include ethenyl, propenyl, cyclohexenyl, and the like.

As used herein, “alkynyl” refers to an alkyl group having one or more triple carbon-carbon bonds. Example alkynyl groups include ethynyl, propynyl, and the like.

As used herein, “haloalkyl” refers to an alkyl group having one or more halogen substituents. Example haloalkyl groups include CF₃, C₂F₅, CHF₂, CCl₃, CHCl₂, C₂Cl₅, and the like. An alkyl group in which all of the hydrogen atoms are replaced with halogen atoms can be referred to as “perhaloalkyl.”

As used herein, “carbocyclyl” refers to groups that are saturated (i.e., containing no double or triple bonds) or unsaturated (i.e., containing one or more double or triple bonds) cyclic hydrocarbon moieties. Carbocyclyl groups can be mono- or polycyclic. Example carbocyclyl groups include cyclopropyl, cyclobutyl, cyclopentyl, cyclohexyl, cycloheptyl, cyclopentenyl, 1,3-cyclopentadienyl, cyclohexenyl, norbornyl, norpinyl, norcarnyl, adamantyl, phenyl, and the like. Carbocyclyl groups can be aromatic (e.g., “aryl”) or non-aromatic (e.g., “cycloalkyl”). In some embodiments, carbocyclyl groups can have from 3 to about 20, 3 to about 10, or 3 to about 7 carbon atoms.

As used herein, “aryl” refers to monocyclic or polycyclic aromatic hydrocarbons such as, for example, phenyl, naphthyl, anthracenyl, phenanthrenyl, indanyl, indenyl, and the like. In some embodiments, aryl groups have from 6 to about 20 carbon atoms.

As used herein, “cycloalkyl” refers to non-aromatic hydrocarbons including cyclized alkyl, alkenyl, and alkynyl groups. Cycloalkyl groups can include mono-, bi- or poly-cyclic ring systems as well as double and triple bonds. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e., having a bond in common with) to the cycloalkyl ring, for example, benzo derivatives of pentane, hexane, and the like.

As used herein, “heterocyclyl” refers to a group that can be a saturated or unsaturated carbocyclyl group wherein one or more of the ring-forming carbon atoms of the carbocyclyl group is replaced by a heteroatom such as O, S, or N. Heterocyclyl groups can be aromatic (e.g., “heteroaryl”) or non-aromatic (e.g., “heterocycloalkyl”). Heterocyclyl groups can correspond to hydrogenated and partially hydrogenated heteroaryl groups. Heterocarbocyclyl groups can contain, in addition to at least one heteroatom, from about 1 to about 20, about 2 to about 10, or about 2 to about 7 carbon atoms and can be attached through a carbon atom or heteroatom. In some embodiments, heterocyclyl groups can have from 3 to 20, 3 to 10, 3 to 7, or 5 to 7 ring-forming atoms. Further, heterocyclyl groups can be substituted or unsubstituted. Examples of heterocyclyl groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like as well as any of the groups listed for heteroaryl and heterocycloalkyl.

As used herein, “heteroaryl” groups are monocyclic and polycyclic aromatic hydrocarbons that have at least one heteroatom ring member such as sulfur, oxygen, or nitrogen. Heteroaryl groups include, without limitation, pyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, 2,3-dihydrobenzofuranyl, 2,3-dihydrobenzothienyl, 2,3-dihydrobenzothienyl-5-oxide, 2,3-dihydrobenzothienyl-5-dioxide, benzoxazolin-2-on-yl, indolinyl, benzodioxolanyl, benzodioxane, and the like. In some embodiments, heteroaryl groups can have from 1 to about 20 carbon atoms, and in further embodiments from about 3 to about 20 carbon atoms. In some embodiments, heteroaryl groups have 1 to about 4, 1 to about 3, or 1 to 2 heteroatoms.

As used herein, “heterocycloalkyl” refers to a cycloalkyl group wherein one or more of the ring-forming carbon atoms is replaced by a heteroatom such as an O, S, N, or P atom. Also included in the definition of heterocycloalkyl are moieties that have one or more aromatic rings fused (i.e., having a bond in common with) to the nonaromatic heterocyclic ring, for example phthalimidyl, naphthalimidyl pyromellitic diimidyl, phthalanyl, and benzo derivatives of saturated heterocycles such as indolene and isoindolene groups.

As used herein, “halo” or “halogen” includes fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like.

As used herein, “haloalkoxy” refers to alkoxy substituted by at least one halo.

As used herein, “thioalkoxy” refers to an alkoxy group in which the O atom is replaced by an S atom.

As used herein, “halothioalkoxy” refers to thioalkoxy substituted by at least one halo.

As used herein, “acyl” refers to a carbonyl group substituted by H, alkyl, alkenyl, alkynyl or carbocyclyl. Example acyl groups include formyl or acetyl.

As used herein, “acyloxy” refers to —O-acyl.

As used herein, “carboxamide” or “aminocarbonyl” refers to —C(O)NH₂.

As used herein, “alkylcarboxamide” or “alkylaminocarbonyl” refers to —C(O)NH(alkyl).

As used herein, “dialkylcarboxamide” or “dialkylaminocarbonyl” refers to —C(O)N(alkyl)₂

As used herein, “sulfonamide” refers to —S(O)NH₂.

As used herein, “alkylsulfonamide” refers to —S(O)NH(alkyl).

As used herein, “dialkylsulfonamide” refers to —S(O)N(alkyl)₂.

As used herein, “sulfonyl” refers to SO2.

As used herein, “sulfinyl” refers to SO.

As used herein, “alkylsulfinyl” refers to sulfinyl substituted by alkyl.

As used herein, “haloalkylsufinyl” refers to sulfinyl substituted by haloalkyl.

As used herein, “arylsulfinyl” refers to sulfinyl substituted by aryl.

As used herein, “alkylsulfonyl” refers to sulfonyl substituted by alkyl.

As used herein, “haloalkylsulfonyl” refers to sulfonyl substituted by haloalkyl.

As used herein, “arylsulfonyl” refers to sulfonyl substituted by aryl.

As used herein, “uerido” refers to —NHC(O)NH₂.

As used herein, “alkyluserido” refers to ureido substituted by an alkyl group.

As used herein, “amino” refers to NH2.

As used herein, “alkylamino” refers to amino substituted by alkyl.

As used herein, “dialkylamino” refers to amino substituted by two alkyl groups.

As used herein, “alkoxycarbonyl” refers to —CO-(alkoxy).

As used herein, “haloalkoxycarbonyl” refers to —CO-(haloalkoxy).

As used herein, “carbocyclylalkyl” refers to alkyl substituted by carbocyclyl.

As used herein, “arylalkyl” refers to an alkyl moiety substituted by an aryl group. Example aralkyl groups include benzyl, phenethyl, and naphthylmethyl groups. In some embodiments, arylalkyl groups have from 7 to 20 or 7 to 11 carbon atoms.

As used herein, “heterocyclylalkyl” refers to alkyl substituted by heterocyclyl.

As used herein, “heterocycloalkylalkyl” refers to alkyl substituted by heterocycloalkyl.

As used herein, the term “reacting” is used as known in the art and generally refers to the bringing together of chemical reagents in such a manner so as to allow their interaction at the molecular level to achieve a chemical or physical transformation of at least one chemical reagent.

As used herein, the term “substituted” refers to the replacement of a hydrogen moiety with a non-hydrogen moiety in a molecule or group.

As used herein, the term “leaving group” refers to a moiety that can be displaced by another moiety, such as by nucleophilic attack, during a chemical reaction. Leaving groups are well known in the art and include, for example, halogen, hydroxy, alkoxy, —O(CO)R^{a}, —OSO₂—R^{b}, and —Si(R)₃ wherein R^{a} can be C₁-C₈ alkyl, C₃-C₇ cycloalkyl, aryl, heteroaryl, or heterocycloalkyl, wherein R^{b} can be C₁-C₈ alkyl, aryl (optionally substituted by one or more halo, cyano, nitro, C₁-C₄ alkyl, C₁-C₄ haloalkyl, C₁-C₄ alkoxy, or C₁-C₄haloalkoxy), or heteroaryl (optionally substituted by one or more halo, cyano, nitro, C₁-C₄ alkyl, C₁-C₄ haloalkyl, C₁-C₄ alkoxy, or C₁-C₄ haloalkoxy), and wherein R^{c} can be C₁-C₈ alkyl. Example leaving groups include chloro, bromo, iodo, mesylate, tosylate, trimethylsilyl, and the like.

As used herein, the term “amino protecting group” refers to a non-hydrogen amino substituent that reversibly preserves a reactively susceptible amino functionality while reacting other functional groups on the compound. A “cyclic amino protecting group” refers to an amino protecting group that includes the protected amino moiety in a ring, such as a phthalimido group, or the like. Examples of amino-protecting groups include formyl, acetyl, trityl, trichloroacetyl, chloroacetyl, bromoacetyl, iodoacetyl, and urethane-type blocking groups such as benzyloxycarbonyl, 4-phenyl-benzyloxycarbonyl, 2-methylbenzyloxycarbonyl, 4-methoxy-benzyloxycarbonyl, 4-fluoro-benzyloxycarbonyl, 4-chloro-benzyloxycarbonyl, 3-chloro-benzyloxycarbonyl, 2-chloro-benzyloxycarbonyl, 2,4-dichloro-benzyloxycarbonyl, 4-bromo-benzyloxycarbonyl, 3-bromo-benzyloxycarbonyl, 4-nitro-benzyloxycarbonyl, 4-cyano-benzyloxycarbonyl, t-butoxycarbonyl, 2-(4-xenyl)-isopropoxycarbonyl, 1,1-diphenyleth-1-yloxycarbonyl, 1,1-diphenylprop-1-yloxycarbonyl, 2-phenylprop-2-yloxycarbonyl, 2-(p-tolyl)-prop-2-yloxycarbonyl, cyclopentanyloxycarbonyl, 1-methyl-cyclopentanyloxycarbonyl, cyclohexanyloxycarbonyl, 1-methylcyclohexanyloxycarbonyl, 2-methylcyclohexanyloxycarbonyl, 2-(4-toluoylsulfonyl)-ethoxycarbonyl, 2-(methylsulfonyl)ethoxycarbonyl, 2-(triphenylphosphino)-ethoxycarbonyl, fluorenylmethoxycarbonyl (FMOC), 2-(trimethylsilyl)ethoxycarbonyl, allyloxycarbonyl, 1-(trimethylsilylmethyl)prop-1-enyloxycarbonyl, 5-benzisoxalylmethoxy-carbonyl, 4-acetoxybenzyloxycarbonyl, 2,2,2-trichloroethoxycarbonyl, 2-ethynyl-2-propoxycarbonyl, cyclopropylmethoxycarbonyl, 4-(decycloxy)benzyloxycarbonyl, isobornyloxy-carbonyl, 1-piperidyloxycarbonyl and the like; benzoylmethylsulfonyl group, 2-nitrophenylsulfenyl, diphenylphosphine oxide and like amino-protecting groups. The species of amino-protecting group employed is not critical so long as the derivatized amino group is stable to the condition of subsequent reaction(s) on other positions of the intermediate molecule and can be selectively removed at the appropriate point without disrupting the remainder of the molecule. In some embodiments, the amino-protecting groups are t-butoxycarbonyl (t-Boc), allyloxycarbonyl and benzyloxycarbonyl (CbZ). In further embodiment, the amino protecting group is an acyl group such as formyl or acetyl. Further examples of amino protecting groups are found in E. Haslam, Protecting Groups in Organic Chemistry, (J. G. W. McOmie, ed., 1973), at Chapter 2; T. W. Greene and P. G. M. Wuts, Protective Groups in Organic Synthesis, (1991), at Chapter 7; and T. W. Greene and P. G. M. Wuts, Protective Groups in Organic Synthesis, 3rd Ed., (1999), at Chapter 7.

As used herein, the phrase “substantially undetectable amount” refers to an amount of compound that is either absent from a composition or present in the composition in an amount that is either not detectable by routine analytical means or is detected in an amount less than about 0.5 mole % compared with the major component of the composition.

The processes described herein can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

In some embodiments, preparation of compounds can involve the protection and deprotection of various chemical groups. The need for protection and deprotection, and the selection of appropriate protecting groups can be readily determined by one skilled in the art. The chemistry of protecting groups can be found, for example, in Greene and Wuts, et al., Protective Groups in Organic Synthesis, 3rd Ed., Wiley & Sons, 1999, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected. In some embodiments, reactions can be carried out in the absence of solvent, such as when at least one of the reagents is a liquid or gas.

Suitable solvents can include halogenated solvents such as carbon tetrachloride, bromodichloromethane, dibromochloromethane, bromoform, chloroform, bromochloromethane, dibromomethane, butyl chloride, dichloromethane, tetrachloroethylene, trichloroethylene, 1,1,1-trichloroethane, 1,1,2-trichloroethane, 1,1-dichloroethane, 2-chloropropane, hexafluorobenzene, 1,2,4-trichlorobenzene, o-dichlorobenzene, chlorobenzene, fluorobenzene, fluorotrichloromethane, chlorotrifluoromethane, bromotrifluoromethane, carbon tetrafluoride, dichlorofluoromethane, chlorodifluoromethane, trifluoromethane, 1,2-dichlorotetrafluorethane and hexafluoroethane.

Suitable ether solvents include: dimethoxymethane, tetrahydrofuran, 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol dimethyl ether, anisole, or t-butyl methyl ether.

Suitable protic solvents can include, by way of example and without limitation, water, methanol, ethanol, 2-nitroethanol, 2-fluoroethanol, 2,2,2-trifluoroethanol, ethylene glycol, 1-propanol, 2-propanol, 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, or glycerol.

Suitable aprotic solvents can include, by way of example and without limitation, tetrahydrofuran (THF), dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethyl sulfoxide, propionitrile, ethyl formate, methyl acetate, hexachloroacetone, acetone, ethyl methyl ketone, ethyl acetate, sulfolane, N,N-dimethylpropionamide, tetramethylurea, nitromethane, nitrobenzene, or hexamethylphosphoramide.

Suitable hydrocarbon solvents include benzene, cyclohexane, pentane, hexane, toluene, cycloheptane, methylcyclohexane, heptane, ethylbenzene, m-, o-, or p-xylene, octane, indane, nonane, or naphthalene.

Supercritical carbon dioxide can also be used as a solvent.

The reactions of the processes described herein can be carried out at appropriate temperatures which can be readily determined by the skilled artisan. Reaction temperatures will depend on, for example, the melting and boiling points of the reagents and solvent, if present; the thermodynamics of the reaction (e.g., vigorously exothermic reactions may need to be carried out at reduced temperatures); and the kinetics of the reaction (e.g., a high activation energy barrier may need elevated temperatures). “Elevated temperature” refers to temperatures above room temperature (about 25° C.) and “reduced temperature” refers to temperatures below room temperature.

The reactions of the processes described herein can be carried out in air or under an inert atmosphere. Typically, reactions containing reagents or products that are substantially reactive with air can be carried out using air-sensitive synthetic techniques that are well known to the skilled artisan.

In some embodiments, preparation of compounds can involve the addition of acids or bases to effect, for example, catalysis of a desired reaction or formation of salt forms such as acid addition salts.

Example acids can be inorganic or organic acids. Inorganic acids include hydrochloric acid, hydrobromic acid, sulfuric acid, phosphoric acid, and nitric acid. Organic acids include formic acid, acetic acid, propionic acid, butanoic acid, methanesulfonic acid, p-toluene sulfonic acid, benzenesulfonic acid, trifluoroacetic acid, propiolic acid, butyric acid, 2-butynoic acid, vinyl acetic acid, pentanoic acid, hexanoic acid, heptanoic acid, octanoic acid, nonanoic acid and decanoic acid.

Example bases include lithium hydroxide, sodium hydroxide, potassium hydroxide, lithium carbonate, sodium carbonate, and potassium carbonate. Some example strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include sodium and potassium salts of methyl, ethyl, n-propyl, i-propyl, n-butyl, t-butyl, trimethylsilyl and cyclohexyl substituted amides.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis.

The processes described herein can be stereoselective such that any given reaction starting with one or more chiral reagents enriched in one stereoisomer forms a product that is also enriched in one stereoisomer. The reaction can be conducted such that the product of the reaction substantially retains one or more chiral centers present in the starting materials. The reaction can also be conducted such that the product of the reaction contains a chiral center that is substantially inverted relative to a corresponding chiral center present in the starting materials.

Resolution of racemic mixtures of compounds can be carried out by any of numerous methods known in the art. An example method includes fractional recrystallization (for example, diastereomeric salt resolution) using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of β-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds of the invention can also include all isotopes of atoms occurring in the intermediates or final compounds. Isotopes include those atoms having the same atomic number but different mass numbers. For example, isotopes of hydrogen include tritium and deuterium.

Compounds of the invention can also include tautomeric forms, such as keto-enol tautomers. Tautomeric forms can be in equilibrium or sterically locked into one form by appropriate substitution.

The present invention also includes salt forms of the compounds described herein. Examples of salts (or salt forms) include, but are not limited to, mineral or organic acid salts of basic residues such as amines, alkali or organic salts of acidic residues such as carboxylic acids, and the like. Generally, the salt forms can be prepared by reacting the free base or acid with stoichiometric amounts or with an excess of the desired salt-forming inorganic or organic acid or base in a suitable solvent or various combinations of solvents. Lists of suitable salts are found in Remington's Pharmaceutical Sciences, 17th ed., Mack Publishing Company, Easton, Pa., 1985, p. 1418, the disclosure of which is hereby incorporated by reference in its entirety.

Upon carrying out preparation of compounds according to the processes described herein, the usual isolation and purification operations such as concentration, filtration, extraction, solid-phase extraction, recrystallization, chromatography, and the like may be used, to isolate the desired products.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

### Preparation of 1-[3-(4-bromo-2-methyl-2H-pyrazol-3-yl)-4-methoxy-phenyl]-3-(2,4-difluoro-phenyl)-urea from N-[3-(4-bromo-2-methyl-2H-pyrazol-3-yl)-4-methoxy-phenyl]-acetamide (Base Hydrolysis Method)

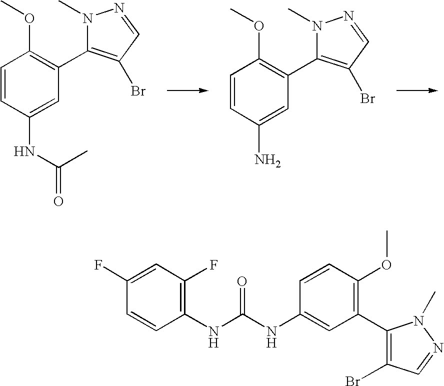

After a stirred mixture of methanol (90 mL), 50 wt % aqueous NaOH (61.60 g, ca. 40.4 mL, 0.7705 mole, 4.993 equivalents), and N-[3-(4-bromo-2-methyl-2H-pyrazol-3-yl)-4-methoxy-phenyl]-acetamide (50.0 g, 0.1542 moles, 1.000 equivalent) had been heated under nitrogen with a 90° C. oil bath for 8.5 hr, the conversion of N-[3-(4-bromo-2-methyl-2H-pyrazol-3-yl)-4-methoxy-phenyl]-acetamide to 3-(4-bromo-2-methyl-2H-pyrazol-3-yl)-4-methoxy-phenylamine was found by HPLC to be 99.6%. Substantially all the methanol was then removed by distillation at reduced pressure. While maintaining the stirred residue at less than 50° C., water (150 mL) and then conc. aqueous HCl (64 mL) were added to achieve a neutral pH of 7. Upon stirring at room temperature for 2 h, product 3-(4-bromo-2-methyl-2H-pyrazol-3-yl)-4-methoxy-phenylamine precipitated as a light tan solid, which was collected by suction filtration, washed with water (2×75 mL), air dried for two hours, and then dissolved in n-propanol (240 mL). 2,4-Difluorophenylisocyanate (19.5 g, 0.12572 mole, 0.815 equivalents) was added to the solution of crude 3-(4-bromo-2-methyl-2H-pyrazol-3-yl)-4-methoxy-phenylamine at a rate sufficiently slow to maintain the stirred reaction mixture at 40-50° C. with cooling. After the addition had been completed, stirring at that temperature was continued for 30 minutes, at which time conversion of 3-(4-bromo-2-methyl-2H-pyrazol-3-yl)-4-methoxy-phenylamine to 1-[3-(4-bromo-2-methyl-2H-pyrazol-3-yl)-4-methoxy-phenyl]-3-(2,4-difluoro-phenyl)-urea was found by HPLC to be 98%. To improve stirrability, acetone (70 mL) and water (300 mL) were added. The resulting mixture was heated to 75° C. and then filtered. The filtered solid is washed with water and dried to provide 1-[3-(4-bromo-2-methyl-2H-pyrazol-3-yl)-4-methoxy-phenyl]-3-(2,4-difluoro-phenyl)-urea.

### Example 2

### Preparation of 1-[3-(4-bromo-2-methyl-2H-pyrazol-3-yl)-4-methoxy-phenyl]-3-(2,4-difluoro-phenyl)-urea from N-[3-(4-bromo-2-methyl-2H-pyrazol-3-yl)-4-methoxy-phenyl]-acetamide (Telescoping by Extraction)

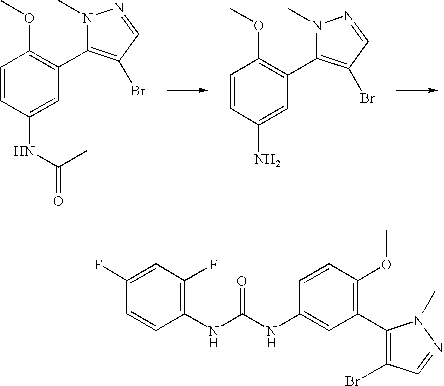

A mixture of methanol (9 mL), 50 wt % aqueous NaOH (6.16 g, 4.04 mL @ d=1.525 g/mL, 77 mmol, 5 equiv.) and N-[3-(4-bromo-2-methyl-2H-pyrazol-3-yl)-4-methoxy-phenyl]-acetamide (5 g, 15.4 mmol, 1 equiv.) was stirred under nitrogen and heated with a 90° C. oil-bath. LC/MS analysis revealed conversions of N-[3-(4-bromo-2-methyl-2H-pyrazol-3-yl)-4-methoxy-phenyl]-acetamide to 3-(4-bromo-2-methyl-2H-pyrazol-3-yl)-4-methoxy-phenylamine of 86.4% after 3.5 h and 99.5% after an additional hour. After a total heating period of 5.5 h at 90° C., methanol was distilled off the reaction mixture under reduced pressure, and the residue was diluted with water (20 mL). The aqueous mixture was then extracted with toluene three times (30 mL, 20 mL, and 15 mL). The toluene layers were combined and washed with water (4×15 mL) until the aqueous wash was neutral (pH 7). The toluene solution was filtered and concentrated under reduced pressure until about 12-15 mL toluene remained with the product [3-(4-bromo-2-methyl-2H-pyrazol-3-yl)-4-methoxy-phenylamine, theoretical yield of 4.35 g, 15.4 mmol]. After n-propanol (35 mL) was added to the crude 3-(4-bromo-2-methyl-2H-pyrazol-3-yl)-4-methoxy-phenylamine, 2,4-difluorophenyl isocyanate (2.62 g, 2.00 mL, 16.9 mmol, 1.1 equiv.) was added dropwise while the stirred reaction mixture was maintained at 0-5° C. with cooling. The mixture was then allowed to warm to room temperature. After approximately 15 min., a solid started to precipitate. n-Propanol (10 mL) added to facilitate stirring, and the resulting mixture was filtered. The filtered white solid was washed with n-propanol (10 mL) and dried overnight at 60° C. at about 20 torr to provide 1-[3-(4-bromo-2-methyl-2H-pyrazol-3-yl)-4-methoxy-phenyl]-3-(2,4-difluoro-phenyl)-urea 4.25 g (63%). HPLC purity, 99.05 (by peak area). 1H NMR (Bruker 400 MHz, DMSO-d₆) δ 9.01 (s, 1H, NH), 8.45 (1H, NH), 8.05 (m, 1H, ArH), 7.61 (s, 1H, ArH), 7.53 (dd, 1H, J=3 Hz, 9 Hz, ArH), 7.36 (d, 1H, J=3 Hz, ArH), 7.30 (m, 1H, ArH), 7.16 (d, 1H, J=9 Hz), 7.08 (m, 1H, ArH), 3.77 (s, 3H), 3.63 (s, 3H).

### Example 3

### Preparation of 1-[3-(4-bromo-2-methyl-2H-pyrazol-3-yl)-4-methoxy-phenyl]-3-(2,4-difluoro-phenyl)-urea from N-[3-(4-bromo-2-methyl-2H-pyrazol-3-yl)-4-methoxy-phenyl]-acetamide

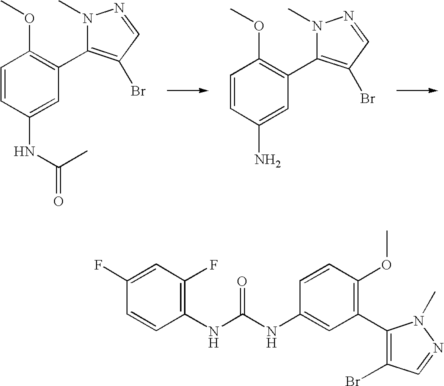

To a mixture of N-[3-(4-bromo-2-methyl-2H-pyrazol-3-yl)-4-methoxy-phenyl]-acetamide (34.7 g, 0.1 mol) in methanol (347 mL) was added acetyl chloride (3 molar equivalents, 23 mL, 0.32 mol) at 0° C. and the solution was stirred at 45° C. for 24 h. Formation of 3-(4-bromo-2-methyl-2H-pyrazol-3-yl)-4-methoxy-phenylamine and consumption of N-[3-(4-bromo-2-methyl-2H-pyrazol-3-yl)-4-methoxy-phenyl]-acetamide were monitored by LCMS. The volatiles were removed, and the resulting residue was dissolved back in methanol (350 mL). Diisopropylethylamine (DIEA) (3 molar equivalents, 56.1 mL, 0.32 mol) was added at room temperature, and, after 0.5 h isocyanate (1.1 molar equivalents, 12.73 mL, 0.101 mol) was then introduced at room temperature. Formation of 1-[3-(4-bromo-2-methyl-2H-pyrazol-3-yl)-4-methoxy-phenyl]-3-(2,4-difluoro-phenyl)-urea and consumption of 3-(4-bromo-2-methyl-2H-pyrazol-3-yl)-4-methoxy-phenylamine were monitored by LCMS, and, after 3 hr, the mixture was heated to 80° C. and diluted using water (70 mL). The white solid product was filtered, washed using water (70 mL), and dried to provide 1-[3-(4-bromo-2-methyl-2H-pyrazol-3-yl)-4-methoxy-phenyl]-3-(2,4-difluoro-phenyl)-urea (32.46 g, 74.28 mmol, 69% yield).

### Example 4

### Preparation of 1-[3-(4-bromo-2-methyl-2H-pyrazol-3-yl)-4-methoxy-phenyl]-3-(2,4-difluoro-phenyl)-urea from N-[3-(4-bromo-2-methyl-2H-pyrazol-3-yl)-4-methoxy-phenyl]-acetamide

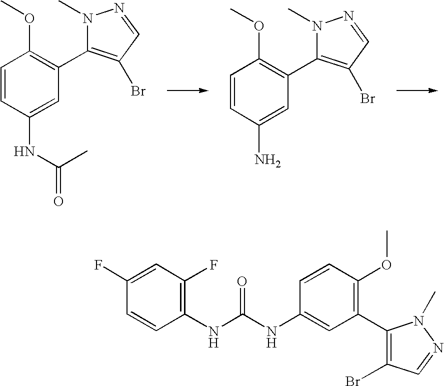

To a mixture of N-[3-(4-bromo-2-methyl-2H-pyrazol-3-yl)-4-methoxy-phenyl]-acetamide (3 g, 9.25 mmol) in 1-propanol (3 mL) and water (6 mL) was added sulfuric acid (2 molar equivalents, 1.81 g, 18.51 mmol) at room temperature and the solution was stirred at 102° C. for 5 h. Formation of 3-(4-bromo-2-methyl-2H-pyrazol-3-yl)-4-methoxy-phenylamine and consumption of N-[3-(4-bromo-2-methyl-2H-pyrazol-3-yl)-4-methoxy-phenyl]-acetamide were monitored by LCMS. Potassium carbonate (2.2 molar equivalents, 2.81 g, 20.36 mmol) was added, the resulting mixture was stirred for 0.5 h, and the solid salts were removed by filtration and washed using 1-propanol (6 mL). After 0.5 h, isocyanate (1.3 molar equivalents, 1.44 mL, 12.01 mmol) was then introduced at room temperature to the combined filtrates. Formation of 1-[3-(4-bromo-2-methyl-2H-pyrazol-3-yl)-4-methoxy-phenyl]-3-(2,4-difluoro-phenyl)-urea and consumption of 3-(4-bromo-2-methyl-2H-pyrazol-3-yl)-4-methoxy-phenylamine were monitored by LCMS, and, after 3 hr, the mixture was heated to 80° C. and diluted using water (42 mL) and acetone (12 mL). The white solid was filtered, washed using a mixture of water/1-propanol (ratio 1/1, 3×60 mL) and dried to provide 1-[3-(4-bromo-2-methyl-2H-pyrazol-3-yl)-4-methoxy-phenyl]-3-(2,4-difluoro-phenyl)-urea (2.89 g, 6.62 mmol, 71% yield).

### Example 5

### Preparation of 1-[3-(4-bromo-2-methyl-2H-pyrazol-3-yl)-4-methoxy-phenyl]-3-(2,4-difluoro-phenyl)-urea from N-[4-methoxy-3-(2-methyl-2H-pyrazol-3-yl)-phenyl]-acetamide

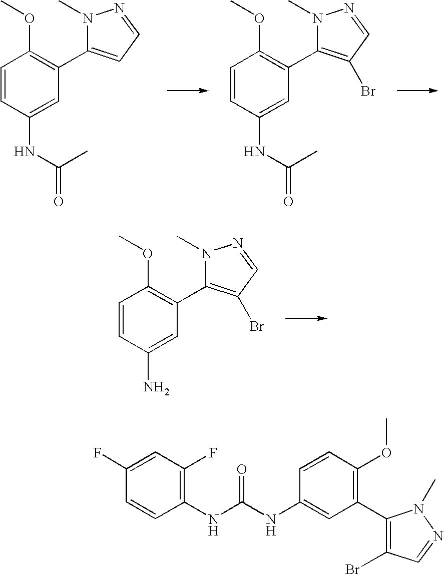

To a mixture of N-[4-methoxy-3-(2-methyl-2H-pyrazol-3-yl)-phenyl]-acetamide (1 g, 4.08 mmol) in methanol (5 mL) was added NBS (1.2 molar equivalent, 871 mg, 4.9 mmol) and the resulting mixture was stirred at room temperature for 2 h. Formation of N-[3-(4-bromo-2-methyl-2H-pyrazol-3-yl)-4-methoxy-phenyl]-acetamide and consumption of N-[4-methoxy-3-(2-methyl-2H-pyrazol-3-yl)-phenyl]-acetamide were monitored by LCMS. The crude mixture was cooled to 0° C., and acetyl chloride (6 molar equivalents, 1.32 mL, 24.28 mmol) was added. The resulting mixture was stirred at 45° C. for 24 h while formation of 3-(4-bromo-2-methyl-2H-pyrazol-3-yl)-4-methoxy-phenylamine and consumption of N-[3-(4-bromo-2-methyl-2H-pyrazol-3-yl)-4-methoxy-phenyl]-acetamide were monitored by LCMS. The volatiles were removed, and the resulting residue was dissolved back in methanol (5 mL). DIEA (3 molar equivalents, 2.13 mL, 12.24 mmol) was added at room temperature and after 0.5 h, isocyanate (1.1 molar equivalents, 0.53 mL, 4.49 mmol) was then introduced at room temperature. Formation of 1-[3-(4-bromo-2-methyl-2H-pyrazol-3-yl)-4-methoxy-phenyl]-3-(2,4-difluoro-phenyl)-urea and consumption of 3-(4-bromo-2-methyl-2H-pyrazol-3-yl)-4-methoxy-phenylamine were monitored by LCMS, and, after 3 hr, the mixture was diluted using water (2 mL). The white solid product was filtered, washed using water (10 mL), and dried to provide 1-[3-(4-bromo-2-methyl-2H-pyrazol-3-yl)-4-methoxy-phenyl]-3-(2,4-difluoro-phenyl)-urea (1.55 g, 3.43 mmol, 84% yield).

### Example 6

### Preparation of N-[3-(4-bromo-2-methyl-2H-pyrazol-3-yl)-4-methoxy-phenyl]-acetamide from N-[4-methoxy-3-(2-methyl-2H-pyrazol-3-yl)-phenyl]-acetamide

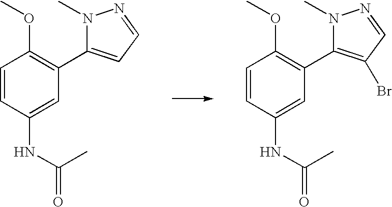

To a solution of N-[4-methoxy-3-(2-methyl-2H-pyrazol-3-yl)-phenyl]-acetamide (5 g, 20.41 mmol) in 15 mL of DMA was added NBS (1.2 molar equivalents, 4.33 g, 24.49 mmol), the resulting mixture was stirred at room temperature for 2.5 hours (the consumption of N-[4-methoxy-3-(2-methyl-2H-pyrazol-3-yl)-phenyl]-acetamide and the formation of 413 were monitored using LCMS). The crude mixture was then diluted using a 0.6N HCl aqueous solution (45 mL), the solid was filtered, washed using water (2×10 mL) and dried. The desired product, N-[3-(4-bromo-2-methyl-2H-pyrazol-3-yl)-4-methoxy-phenyl]-acetamide, was collected as a light tan solid (6.26 g, 19.3 mmol, 94.6%). ¹H NMR (400 MHz, δ ppm, DMSO-d₆): 9.95 (1H, s), 7.7 (1H, dd), 7.6 (1H, s), 7.48 (1H, d), 7.15 (1H, d), 3.76 (3H, s), 3.61 (3H, s), and 2.02 (3H, s). LCMS: 324/326 (MH+), 309.2, 245, 230.1, 203.1, 188.2, and 172.1.

Using essentially a similar synthetic procedure with 12 Kg of N-[4-methoxy-3-(2-methyl-2H-pyrazol-3-yl)-phenyl]-acetamide resulted in the desired product, N-[3-(4-bromo-2-methyl-2H-pyrazol-3-yl)-4-methoxy-phenyl]-acetamide (yield 14 Kg) with a purity of 99.62 and very little if any desbromo compound, N-[4-methoxy-3-(2-methyl-2H-pyrazol-3-yl)-phenyl]-acetamide, <0.05% as determined by HPLC.

HPLC Conditions:

Column: Waters Symmetry Shield C18, 3.5 μm, 4.6×150 mm with a pre-column filter or equivalent filter; Mobile Phase: A=Deionized Water; Mobile Phase: B=HPLC Grade ACN; Autosampler Rinse: HPLC Grade ACN; Flow Rate: 1.5 mL/min; Column Temperature: 45° C.; Autosampler Temperature Ambient; Detector Wavelength: 245 nm; Sample Injection Volume: 10 μL;

Gradient Profile:

| Time | Flow | | | |
|---|---|---|---|---|
| (min.) | (mL/min.) | % A | % B | |
| 0 | 1.50 | 80.0 | 20.0 | — |
| 10.0 | 1.50 | 62.0 | 38.0 | 6 |
| 13.0 | 1.50 | 80.0 | 20 | 1 |

Data acquisition time: 10 minutes; Gradient re-equilibration time: 3 minutes

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

1. A process for preparing a compound of Formula (I): 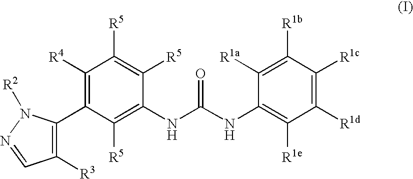
wherein:
R^{1a}, R^{1b}, R^{1c}, R^{1d}, and R^{1e} are each, independently, H, halo, cyano, nitro, C₁₋₆ alkyl, C₁₋₆ haloalkyl, C₂₋₆ alkenyl, C₂₋₆ alkynyl, OR⁷, SR⁷, SOR⁸, SO₂R⁸, COR⁸, COOR⁷, OC(O)R⁸, NR⁹R¹⁰, carbocyclyl optionally substituted by one or more R⁶ or heterocyclyl optionally substituted by one or more R⁶; or R^{1a} and R^{1b}, R^{1b} and R^{1c}, R^{1c} and R^{1d}, or R^{1d} and R^{1e} together with the carbon atoms to which they are attached form a fused C₅₋₇ cycloalkyl group or fused C₅, heterocycloalkyl group; wherein each of said C₁₋₆ alkyl, C₂₋₆ alkenyl, and C₂₋₆ alkynyl, is optionally substituted with one or more C₁₋₆ acyl, C₁₋₆ acyloxy, C₁₋₆ alkoxy, C₁₋₆ thioalkoxy, carboxamide, C₁₋₆ alkylcarboxamide, C₂₋₈ dialkylcarboxamide, C₁₋₆ alkylsulfonamide, C₁₋₆ alkylsulfinyl, C₁₋₆ alkylsulfonyl, C₁₋₆ alkylureido, amino, C₁₋₆ alkylamino, C₂₋₈ dialkylamino, C₁₋₆alkoxycarbonyl, carboxy, cyano, C₃, cycloalkyl, halogen, C₁₋₆ haloalkoxy, C₁₋₆ halothioalkoxy, C₁₋₆ haloalkyl, C₁₋₆ haloalkylsulfinyl, C₁₋₆ haloalkylsulfonyl, hydroxyl, mercapto or nitro;
R² is C₁₋₄ alkyl;
R³ is F, Cl, Br or I;
R⁴ is halo, cyano, nitro, C₁₋₄ alkyl, C₁₋₆ haloalkyl, C₂₋₆ alkenyl, C₂₋₆ alkynyl, C₁₋₆ alkoxy, SR¹¹, SOR¹², SO₂R¹², COR¹², COOR¹¹, OC(O)R¹², NR¹³R¹⁴, or C₃₋₇ cycloalkyl, wherein said C₁₋₆ alkoxy group is optionally substituted with one or more C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ thioalkoxy, C₁₋₄ alkylureido, amino, (C₁₋₆ alkoxy)carbonyl, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ halothioalkoxy, hydroxyl, nitro or phenyl optionally substituted with 1 to 5 halogen atoms;
R⁵, at each independent occurrence, is H, halo, cyano, nitro, C₁₋₆ alkyl, C₁₋₆ haloalkyl, C₂₋₆ alkenyl, C₂₋₆ alkynyl, C₁₋₆ alkoxy, SR¹¹, SOR¹², SO₂R¹², COR¹², COOR¹¹, OC(O)R¹², NR¹³R¹⁴, or C₃₋₇ cycloalkyl, wherein said C₁₋₆ alkoxy group is optionally substituted with one or more C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ thioalkoxy, C₁₋₄ alkylureido, amino, (C₁₋₆ alkoxy)carbonyl, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ halothioalkoxy, hydroxyl, nitro or phenyl optionally substituted with 1 to 5 halogen atoms;
R⁶ is halo, cyano, nitro, C₁₋₄ alkyl, C₁₋₄ haloalkyl, C₁₋₄ alkoxy, C₁₋₄ haloalkoxy, amino, (C₁₋₄ alkyl)amino, di(C₁₋₄ alkyl)amino, hydroxy, carboxy, (C₁₋₄ alkoxy)carbonyl, C₁₋₄ acyl, C₁₋₄ acyloxy, aminocarbonyl, (C₁₋₄ alkyl)aminocarbonyl, or di(C₁₋₄ alkyl)aminocarbonyl;
R⁷ and R¹¹ are each, independently, H, C₁₋₈ alkyl, C₁₋₈ haloalkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl or (5-7 membered heterocycloalkyl)alkyl;
R⁸ and R¹² are each, independently, H, C₁₋₈ alkyl, C₁₋₈ haloalkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl, (5-7 membered heterocycloalkyl)alkyl, amino, (C₁₋₄ alkyl)amino, or di(C₁₋₄ alkyl)amino;
R⁹ and R¹⁰ are each, independently, H, C₁₋₈ alkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl, (5-7 membered heterocycloalkyl)alkyl, (C₁₋₈ alkyl)carbonyl, (C₁₋₈ haloalkyl)carbonyl, (C₁₋₈ alkoxy)carbonyl, (C₁₋₈ haloalkoxy)carbonyl, (C₁₋₄ alkyl)sulfonyl, (C₁₋₄ haloalkyl)sulfonyl or arylsulfonyl;
or R⁹ and R¹⁰, together with the N atom to which they are attached form a 5-7 membered heterocycloalkyl group; and
R¹³ and R¹⁴ are each, independently, H, C₁₋₈ alkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl, (5-7 membered heterocycloalkyl)alkyl, (C₁₋₈ alkyl)carbonyl, (C₁₋₈ haloalkyl)carbonyl, (C₁₋₈ alkoxy)carbonyl, (C₁₋₈ haloalkoxy)carbonyl, (C₁₋₄ alkyl)sulfonyl, (C₁₋₄ haloalkyl)sulfonyl or arylsulfonyl;
or R¹³ and R¹⁴, together with the N atom to which they are attached form a 5-7 membered heterocycloalkyl group;
the process comprising:
a) reacting a compound of Formula (II): 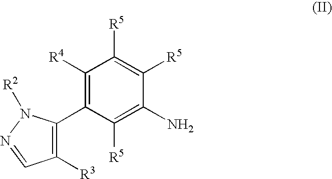
with a compound of Formula (III): 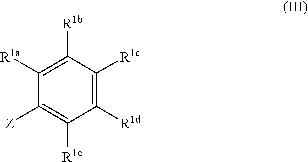
wherein Z is an isocyanate group (—NCO) or isocyanate equivalent, in a Urea Forming C₁₋₈ alcohol solvent for a time and under conditions suitable for forming said compound of Formula (I); or
b) reacting a compound of Formula (II) with an isocyanate-generating reagent for a time and under conditions suitable for forming a compound of Formula (IIa): 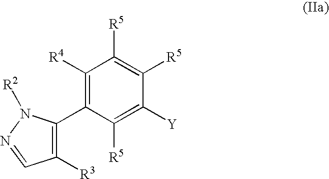
wherein Y is an isocyanate group or isocyanate equivalent; and reacting said compound of Formula (IIa) with a compound of Formula (IIIa): 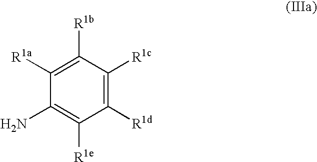
in a Urea Forming C₁₋₈ alcohol solvent for a time and under conditions suitable for forming said compound of Formula (I).

2. The process of claim 1, wherein:
R^{1a} is F;
R^{1b} is H;
R^{1c} is F;
R^{1d} is H;
R^{1e} is H;
R² is methyl;
R³ is Br;
R⁴ is methoxy; and
R⁵, at each occurrence, is H.

3. The process according to claim 1 or 2, wherein said process comprises reacting a compound of Formula (II): 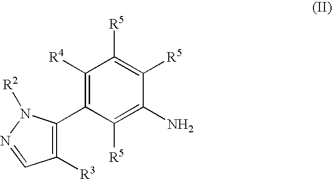
with a compound of Formula (III): 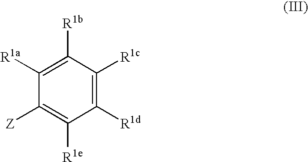
wherein Z is an isocyanate group, in a Urea Forming C₁₋₈ alcohol solvent for a time and under conditions suitable for forming said compound of Formula (I).

4. The process according to claim 1 or 2, wherein said Urea Forming C₁₋₈ alcohol solvent is selected from the group consisting of methanol, ethanol, 1-propanol, 1-butanol and 2-methyl-propan-1-ol.

5. The process according to claim 1 or 2, wherein said Urea Forming C₁₋₈ alcohol solvent is methanol.

6. The process according to claim 1 or 2, wherein said Urea Forming C₁₋₈ alcohol solvent is 1-propanol.

7. The process according to claim 1 or 2, wherein said reacting is carried out at a temperature between about −5° C. to about 75° C.

8. The process according to claim 1 or 2, wherein said compound of Formula (III) is added to a solution containing said compound of Formula (II).

9. The process of claim 1 or 2, wherein said compound of Formula (II) is prepared by the process comprising reacting a compound of Formula (IV): 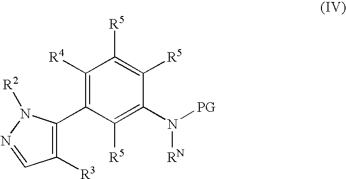
wherein:
PG is an amino protecting group; and
R^{N} is H;
or PG and R^{N} together with the N atom to which they are attached form a cyclic amino protecting group;
with an acid for a time and under conditions suitable for forming said compound of Formula (II).

10. The process of claim 9, wherein PG is —C(O)Me.

11. The process of claim 9, wherein said reacting with an acid is carried out in methanol.

12. The process of claim 9, wherein said reacting with an acid is carried out in 1-propanol.

13. The process according to claim 9, wherein said acid is selected from the group consisting of HCl, HBr, sulfuric acid, methane sulfonic acid, trifluoromethane sulfonic acid and p-toluene sulfonic acid.

14. The process according to claim 9, wherein said acid comprises sulfuric acid.

15. The process according to claim 9, wherein said acid comprises HCl.

16. The process according to claim 9, wherein said reacting with an acid is carried out at a temperature between about 20° C. to about 120° C.

17. The process according to claim 9, wherein said reacting with an acid results in formation of less than about 2 mole % of a compound of Formula (IIb): 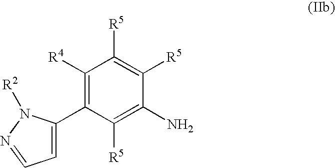
relative to the amount of compound of Formula (II).

18. The process according to claim 9, wherein said reacting with an acid is carried out in a Deprotecting C₁₋₈ alcohol solvent and is essentially the same solvent as said Urea Forming C₁₋₈ alcohol solvent.

19. The process of claim 18, wherein said Deprotecting C₁₋₈ alcohol solvent and Urea Forming C₁₋₈ alcohol solvent both comprise 1-propanol.

20. The process of claim 9, wherein said compound of Formula (IV) is prepared by the process comprising reacting a compound of Formula (V): 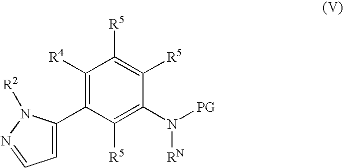
with a Halogenating reagent, in an amide solvent, a Halogenating C₁₋₈ alcohol solvent or mixture thereof, for a time and under conditions suitable for forming said compound of Formula (IV).

21. The process of claim 20, wherein said Halogenating reagent is a brominating reagent.

22. The process of claim 20, wherein said Halogenating reagent comprises N-bromosuccinimide.

23. The process according to claim 20, wherein said reacting with a Halogenating reagent is carried out at a temperature about 30° C. or below.

24. The process according to claim 20, wherein said reacting with a Halogenating reagent results in about 98 mol % conversion or higher of said compound of Formula (IV) compared to said compound of Formula (V) and isolated said compound of Formula (IV) containing about 2 mol % or lower of said compound of Formula (V).

25. The process according to claim 20, wherein said amide solvent is dimethylacetamide.

26. The process according to claim 20, wherein said amide solvent is N-methyl-2-pyrrolidone.

27. The process of claim 1 or 2 wherein said compound of Formula (II) is prepared by the process comprising reacting a compound of Formula (IV): 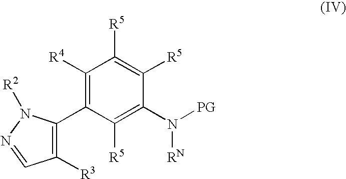
wherein:
PG is an amino protecting group; and
R^{N} is H;
or PG and R^{N} together with the N atom to which they are attached form a cyclic amino protecting group;
with a base for a time and under conditions suitable for forming said compound of Formula (II).

28. The process of claim 27, wherein PG is —C(O)Me.

29. The process of claim 27, wherein said base is sodium hydroxide.

30. The process according to claim 27, wherein said reacting is carried out in a Deprotecting C₁₋₈ alcohol solvent comprising 1-propanol.

31. The process according to claim 30, wherein said Urea Forming C₁₋₈ alcohol solvent comprises 1-propanol.

32. The process according to claim 27, wherein said reacting with a base is carried out at a temperature between about 20° C. to about 120° C.

33. The process according to claim 27, wherein said compound of Formula (II) is isolated containing less than about 2 mole % of a compound of Formula (IIb): 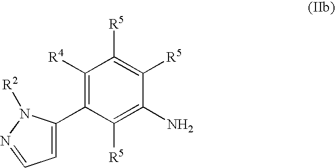
relative to the amount of compound of Formula (II).

34. A process for preparing a compound of Formula (II): 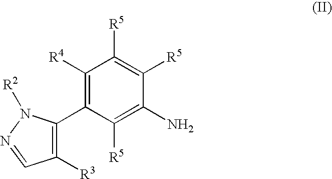
wherein:
R² is C₁₋₄ alkyl;
R³ is F, Cl, Br or I;
R⁴ is halo, cyano, nitro, C₁₋₆ alkyl, C₁₋₄ haloalkyl, C₂₋₆ alkenyl, C₂₋₆ alkynyl, C₁₋₆ alkoxy, SR¹¹, SOR¹², SO₂R¹², COR¹², COOR¹¹, OC(O)R¹², NR¹³R¹⁴, or C₃₋₇ cycloalkyl, wherein said C₁₋₄ alkoxy group is optionally substituted with one or more C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ thioalkoxy, C₁₋₄ alkylureido, amino, (C₁₋₆ alkoxy)carbonyl, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ halothioalkoxy, hydroxyl, nitro or phenyl optionally substituted with 1 to 5 halogen atoms;
R⁵, at each independent occurrence, is H, halo, cyano, nitro, C₁₋₆ alkyl, C₁₋₆ haloalkyl, C₂₋₆ alkenyl, C₂₋₆ alkynyl, C₁₋₆ alkoxy, SR¹¹, SOR¹², SO₂R¹², COR¹², COOR¹¹, OC(O)R¹², NR¹³R¹⁴, or C₃₋₇ cycloalkyl, wherein said C₁₋₆ alkoxy group is optionally substituted with one or more C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C14 thioalkoxy, C₁₋₄ alkylureido, amino, (C₁₋₆ alkoxy)carbonyl, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ halothioalkoxy, hydroxyl, nitro or phenyl optionally substituted with 1 to 5 halogen atoms;
R¹¹ is, independently, H, C₁₋₈ alkyl, C₁₋₈ haloalkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl or (5-7 membered heterocycloalkyl)alkyl;
R¹² is, independently, H, C₁₋₈ alkyl, C₁₋₈ haloalkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl, (5-7 membered heterocycloalkyl)alkyl, amino, (C₁₋₄ alkyl)amino, or di(C₁₋₄ alkyl)amino; and
R¹³ and R¹⁴ are each, independently, H, C₁₋₈ alkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl, (5-7 membered heterocycloalkyl)alkyl, (C₁₋₈ alkyl)carbonyl, (C₁₋₈ haloalkyl)carbonyl, (C₁₋₈ alkoxy)carbonyl, (C₁₋₈ haloalkoxy)carbonyl, (C₁₋₄ alkyl)sulfonyl, (C₁₋₄ haloalkyl)sulfonyl or arylsulfonyl;
or R¹³ and R¹⁴, together with the N atom to which they are attached form a 5-7 membered heterocycloalkyl group;
comprising reacting a compound of Formula (IV): 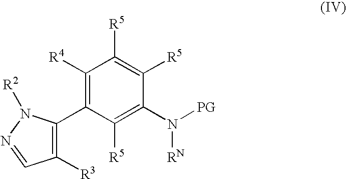
wherein:
PG is an amino protecting group; and
R^{N} is H;
or PG and R^{N} together with the N atom to which they are attached form a cyclic amino protecting group;
with an acid for a time and under conditions suitable for forming said compound of Formula (II).

35. The process of claim 34, wherein:
R² is methyl;
R³ is Br;
R⁴ is methoxy; and
R⁵, at each occurrence, is H.

36. The process of claim 34 or 35, wherein PG is —C(O)—(C₁₋₆ alkyl).

37. The process according to claim 34 or 35, wherein PG is —C(O)Me.

38. The process according to claim 34 or 35, wherein said reacting with an acid is carried out in a 1° alcohol or 2° alcohol.

39. The process according to claim 34 or 35, wherein said reacting with an acid is carried out in a 1° alcohol.

40. The process of claim 39, wherein said 1° alcohol is selected from the group consisting of methanol, ethanol, 1-propanol, 1-butanol and 2-methyl-propan-1-ol.

41. The process of claim 39, wherein said 1° alcohol is methanol.

42. The process of claim 39, wherein said 1° alcohol is 1-propanol.

43. The process according to claim 34 or 35, wherein said acid is selected from the group consisting of HCl, HBr, sulfuric acid, methane sulfonic acid, trifluoromethane sulfonic acid and p-toluene sulfonic acid.

44. The process of claim 43, wherein said acid comprises sulfuric acid.

45. The process of claim 43, wherein said acid comprises HCl.

46. The process of claim 45, wherein the molar ratio of HCl to compound of Formula (IV) is between about 2 to about 4.

47. The process according to claim 34 or 35, wherein said reacting with an acid is carried out at a temperature between about 20° C. to about 120° C.

48. The process according to claim 34 or 35, wherein said reacting with an acid results in formation of less than about 2 mole % of a compound of Formula (IIb): 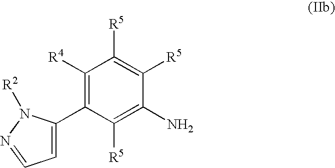
relative to the amount of compound of Formula (II).

49. A process for the preparation of a compound of Formula (IV): 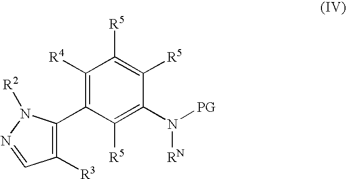
wherein:
R² is C₁₋₄ alkyl;
R³ is F, Cl, Br or I;
R⁴ is halo, cyano, nitro, C₁₋₆ alkyl, C₁₋₆ haloalkyl, C₂₋₆ alkenyl, C₂₋₆ alkynyl, C₁₋₆ alkoxy, SR¹¹, SOR¹², SO₂R¹², COR¹², COOR¹¹, OC(O)R¹², NR¹³R¹⁴, or C₃₋₇ cycloalkyl, wherein said C₁₋₆ alkoxy group is optionally substituted with one or more C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, Cl₄ thioalkoxy, C₁₋₄ alkylureido, amino, (C₁₋₆ alkoxy)carbonyl, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ halothioalkoxy, hydroxyl, nitro or phenyl optionally substituted with 1 to 5 halogen atoms;
R⁵, at each independent occurrence, is H, halo, cyano, nitro, C₁₋₆ alkyl, C₁₋₆ haloalkyl, C₂₋₆ alkenyl, C₂₋₆ alkynyl, C₁₋₆ alkoxy, SR¹¹, SOR¹², SO₂R¹², COR¹², COOR¹¹, OC(O)R¹², NR¹³R¹⁴, or C₃₋₇ cycloalkyl, wherein said C₁₋₆ alkoxy group is optionally substituted with one or more C₁₋₅ acyl, C₁₋₅ acyloxy, C₂₋₆ alkenyl, C₁₋₄ alkoxy, C₁₋₈ alkyl, C₁₋₆ alkylamino, C₂₋₈ dialkylamino, C₁₋₄ alkylcarboxamide, C₂₋₆ alkynyl, C₁₋₄ alkylsulfonamide, C₁₋₄ alkylsulfinyl, C₁₋₄ alkylsulfonyl, C₁₋₄ thioalkoxy, C₁₋₄ alkylureido, amino, (C₁₋₆ alkoxy)carbonyl, carboxamide, carboxy, cyano, C₃₋₆ cycloalkyl, C₂₋₆ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ halothioalkoxy, hydroxyl, nitro or phenyl optionally substituted with 1 to 5 halogen atoms;
R¹¹ is, independently, H, C₁₋₈ alkyl, C₁₋₈ haloalkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl or (5-7 membered heterocycloalkyl)alkyl;
R¹² is, independently, H, C₁₋₈ alkyl, C₁₋₈ haloalkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl, (5-7 membered heterocycloalkyl)alkyl, amino, (C₁₋₄ alkyl)amino, or di(C₁₋₄ alkyl)amino;
R¹³ and R¹⁴ are each, independently, H, C₁₋₈ alkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl, (5-7 membered heterocycloalkyl)alkyl, (C₁₋₈ alkyl)carbonyl, (C₁₋₈ haloalkyl)carbonyl, (C₁₋₈ alkoxy)carbonyl, (C₁₋₈ haloalkoxy)carbonyl, (C₁₋₄ alkyl)sulfonyl, (C₁₋₄ haloalkyl)sulfonyl or arylsulfonyl;
or R¹³ and R¹⁴, together with the N atom to which they are attached form a 5-7 membered heterocycloalkyl group;
PG is an amino protecting group; and
R^{N} is H;
or PG and R^{N} together with the N atom to which they are attached form a cyclic amino protecting group;
comprising reacting a compound of Formula (V): 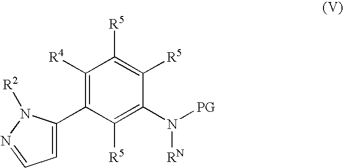
with a halogenating reagent in an amide solvent for a time and under conditions suitable for forming said compound of Formula (IV).

50. The process of claim 49, wherein:
R² is methyl;
R³ is Br;
R⁴ is methoxy; and
R⁵, at each occurrence, is H.

51. The process of claim 49 or 50, wherein said halogenating reagent is a brominating reagent.

52. The process according to claim 49 or 50, wherein said halogenating reagent comprises N-bromosuccinimide.

53. The process according to claim 49 or 50, wherein said amide solvent is dimethylacetamide.

54. The process according to claim 49 or 50, wherein said reacting with a halogenating reagent is carried out at a temperature about 30° C. or below.

55. The process according to claim 49 or 50, wherein said reacting with a halogenating reagent results in about 98% conversion or higher of said compound of Formula (IV) compared to said compound of Formula (V) and isolated said compound of Formula (IV) containing about 2 mol % or lower of said compound of Formula (V).

56. A product prepared by the process of claim 2.

57. The product of claim 56 which is crystalline 1-[3-(4-bromo-2-methyl-2H-pyrazol-3-yl)-4-methoxy-phenyl]-3-(2,4-difluoro-phenyl)-urea.

58. A product prepared by the process of claim 3 which is crystalline 1-[3-(4-bromo-2-methyl-2H-pyrazol-3-yl)-4-methoxy-phenyl]-3-(2,4-difluoro-phenyl)-urea.

59. A product prepared by the process of claim 4 which is crystalline 1-[3-(4-bromo-2-methyl-2H-pyrazol-3-yl)-4-methoxy-phenyl]-3-(2,4-difluoro-phenyl)-urea.

60. A product prepared by the process of claim 5 which is crystalline 1-[3-(4-bromo-2-methyl-2H-pyrazol-3-yl)-4-methoxy-phenyl]-3-(2,4-difluoro-phenyl)-urea.

61. A product prepared by the process of claim 6 which is crystalline 1-[3-(4-bromo-2-methyl-2H-pyrazol-3-yl)-4-methoxy-phenyl]-3-(2,4-difluoro-phenyl)-urea.

62. A compound which is 1-[3-(4-bromo-2-methyl-2H-pyrazol-3-yl)-4-methoxy-phenyl]-3-(2,4-difluoro-phenyl)-urea having a differential scanning calorimetry thermogram comprising an endotherm at about 194° C.

63. The compound of claim 62 having a differential scanning calorimetry thermogram substantially as shown in FIG. 2.

64. A compound which is 1-[3-(4-bromo-2-methyl-2H-pyrazol-3-yl)-4-methoxy-phenyl]-3-(2,4-difluoro-phenyl)-urea having an X-ray powder diffraction pattern comprising at least one peak having a 2-theta value that corresponds to the 2-theta value of the peak of highest intensity in the X-ray powder diffraction pattern of FIG. 1.

65. The compound of claim 64 wherein said compound has an X-ray powder diffraction pattern further comprising at least one peak having a 2-theta value that corresponds to the 2-theta value of the peak of second highest intensity in the X-ray powder diffraction pattern of FIG. 1.

66. The compound of claim 65 wherein said compound has an X-ray powder diffraction pattern further comprising at least one peak having a 2-theta value that corresponds to the 2-theta value of the peak of third highest intensity in the X-ray powder diffraction pattern of FIG. 1.

67. The compound of claim 66 wherein said compound has an X-ray powder diffraction pattern further comprising at least one peak having a 2-theta value that corresponds to the 2-theta value of the peak of fourth highest intensity in the X-ray powder diffraction pattern of FIG. 1.

68. The compound of claim 67 wherein said compound has an X-ray powder diffraction pattern further comprising at least one peak having a 2-theta value that corresponds to the 2-theta value of the peak of fifth highest intensity in the X-ray powder diffraction pattern of FIG. 1.

69. A compound which is 1-[3-(4-bromo-2-methyl-2H-pyrazol-3-yl)-4-methoxy-phenyl]-3-(2,4-difluoro-phenyl)-urea having an X-ray powder diffraction pattern comprising at least two peaks having 2-theta values selected from the 2-theta values of the five peaks of highest intensity in the X-ray powder diffraction pattern of FIG. 1.

70. A compound which is 1-[3-(4-bromo-2-methyl-2H-pyrazol-3-yl)-4-methoxy-phenyl]-3-(2,4-difluoro-phenyl)-urea having an X-ray powder diffraction pattern substantially as shown in FIG. 1.
